(19) United States
(12) Patent Application Publication
(10) Pub. No.: US 2022/0079515 A1
(43) Pub. Date: Mar. 17, 2022

(54) BRAIN CONNECTIVITY MARKER OF SUSTAINED PAIN AND METHOD OF DIAGNOSING SUSTAINED PAIN USING THE SAME

(71) Applicants: RESEARCH & BUSINESS FOUNDATION SUNGKYUNKWAN UNIVERSITY, Suwon-si (KR); THE TRUSTEES OF DARTMOUTH COLLEGE, Hanover, NH (US); INSTITUTE FOR BASIC SCIENCE, Daejeon (KR)

(72) Inventors: Choong-Wan WOO, Suwon-si (KR); Jae-Joong LEE, Suwon-si (KR); Tor D. Wager, Hanover, NH (US)

(73) Assignees: RESEARCH & BUSINESS FOUNDATION SUNGKYUNKWAN UNIVERSITY, Suwon-si (KR); THE TRUSTEES OF DARTMOUTH COLLEGE, Hanover, NH (US); INSTITUTE FOR BASIC SCIENCE, Daejeon (KR)

(21) Appl. No.: 17/473,253

(22) Filed: Sep. 13, 2021

Related U.S. Application Data
(60) Provisional application No. 63/078,498, filed on Sep. 15, 2020.

(30) Foreign Application Priority Data
Mar. 10, 2021 (KR) 10-2021-0031652

(51) Int. Cl. A61B 5/00; A61B 5/055

(52) U.S. Cl. CPC A61B 5/4824; A61B 5/055; A61B 5/0042

(57) ABSTRACT

Disclosed are brain connectivity marker of sustained pain and a method of diagnosing sustained pain using the same. The marker is specific for pain, and does not respond to other noxious stimuli. It can be used for monitoring a pain level and a response to treatment of chronic pain patients, which are considered clinically significant. By comparing a responsive clinical pain group and a non-responsive clinical pain group, the disclosure may be used for differential diagnosis for the cause of pain. The disclosure may be used for pre-screening of a drug clinical trial to dramatically reduce time and costs consumed in trial, and contributes to the development of pain treatment methods such as brain stimulation based on a weight pattern of the marker. The disclosure maybe used to measure a pain level in groups which have difficulty in reporting pain (a vegetative state, aphasia patients, the elderly, infants, etc).

### CROSS-REFERENCE TO RELATED APPLICATION

This application claims priority to and the benefit of U.S. Provisional Patent Application No. 63/073,498, filed on Sep. 15, 2020, and Korean Patent Application No. 10-2021-0031652, filed on Mar. 20, 2021, the disclosure of which is incorporated herein by reference in its entirety.

### BACKGROUND

### 1. Field of the Present Invention

The present invention relates to a brain connectivity marker of sustained pain and a method of diagnosing sustained pain using the same.

### 2. Discussion of Related Art

Most of the existing pain ratings were accomplished in the form of self-reporting of a rating target such as visual analogue scale (VAS) or numerical rating scale (NRS). However, such self-reporting has a limitation in use in groups with difficulty in expressing pain, such as patients with aphasia or in a vegetative state, the elderly or infants. In addition, due to the features of self-reporting, subjective factors which are difficult to quantify are introduced, and pointed out as one of the reasons for why many clinical trials of pain relief drugs fail to show better effects than a placebo group.

With the recent development of magnetic resonance imaging technology, to solve the above problems, a method of measuring the activation and interaction of the brain, which is the organ where pain experiences are made, and developing a pain marker based on this data is attracting attention. Particularly, the pain marker which was developed by Wager and Lindquist is now disclosed (US 20160054409A1), and this marker succeeded in predicting the subjective intensity of heat pain at an individual level based on a brain activation pattern when acute heat pain induced for a short time is felt, showing the possibility of the development of a brain-based pain marker.

However, the marker of the related patent is a marker limited to pain for a short time in units of seconds. One of the most important characteristics of clinical pain is its sustained nature, which may contribute to the involvement of brain regions related to top-down cognitive and affective coping responses. Therefore, the previous marker of the related patent does not in various cognitive/attentive/emotional control and adaptation processes at the brain level, which occur when humans feel unavoidable pain for a long time pointed out as the clinically common mechanism of tonic/chronic pain are not reflected, and for this reason, in practice, the conventional marker has not yet proven predictive power for tonic/chronic pain. In addition, since the conventional marker was modeled based on a brain activation pattern, to test the corresponding marker, a method of predicting the intensity of pain, (1) after specifying the time period at which an experimental pain stimulus is repeatedly given, (2) by quantitatively calculating how much brain activation increases specifically in the corresponding time period in each brain region, and (3) calculating similarity by comparing the degree of brain activation with the conventional marker, is used. However, since many types of clinical pain have the characteristic of spontaneously and continuously fluctuating for a long time, it is very difficult to specify the time period at which pain is induced so that there is a big problem in applying the conventional marker. Finally, while many previous studies have continuously revealed that patient experiencing tonic/chronic pain have a difference from a group which does not experience pain at inter-brain connectivity, the conventional marker is a marker based on a brain activation pattern and has a disadvantage of having no brain connectivity data.

### SUMMARY OF THE PRESENT INVENTION

Therefore, the inventors had tried to develop an objective marker of pain by identifying a neurobiological mechanism of sustained pain, particularly, in the brain, and measure the degree of sustained pain experienced by humans using this marker. Pain is a multi-dimensional experience which is not necessarily proportional to the degree of physical damage and created by the interaction and integration of complicated sensory, emotional and contextual factors in the brain. The present invention is to quantitatively model how numerous brain regions, which are known to be responsible for different functions, interact and are integrated to create a pain experience when there is sustained pain, based on the theoretical background.

Therefore, the present invention is directed to providing a biomarker composition for diagnosing sustained pain, which includes one or more selected from 1 to 39 brain functional connectivity regions, listed in Tables 1 and 2 below.

The present invention is also directed to providing a system for diagnosing sustained pain, which includes:

a receiver for receiving brain image data of a subject;

an analyzer for analyzing one or more selected from 1 to 39 brain functional connectivity regions listed in Tables 1 and 2 below from the received data; and

a calculator for calculating a signature response based on the brain functional connectivity.

TABLE 1

| Rank (# i) | Brain functional connectivity region |
|---|---|
| #1 | Lt. Inferior temporal gyrus (BA37, ventrolateral) - |
| | Lt. middle occipital gyrus |
| #2 | Lt. middle temporal gyrus (BA37, dorsolateral) - |
| | Lt. middle occipital gyrus |
| #3 | Lt. precentral gyrus (BA4, trunk) - Rt. precuneus |
| | (BA7, medial) |
| #4 | Lt. precuneus (BA7, medial) - Lt. postcentral gyrus |
| | (BA1/2/3, trunk) |
| #5 | Rt. inferior parietal lobule (BA40, rostrodorsal) - |
| | Lt. parietooccipital sulcus (dorsomedial) |
| #6 | Rt. precentral gyrus (BA4, upper limb) - Lt. inferior |
| | parietal lobule (BA39, rostrodorsal) |
| #7 | Lt. precentral gyrus (BA4, trunk) - Rt. medial superior |
| | occipital gyrus |
| #8 | Lt. paracentral lobule (BA4, lower limb) - Rt. |
| | precuneus (BA8, medial) |
| #9 | Lt. precentral gyrus (BA4, trunk) - Lt. precuneus (BA8, |
| | medial) |
| #10 | Rt. precuneus (BA7, medial) - Lt. postcentral gyrus |
| | (BA1/2/3, trunk) |
| #11 | Lt. superior temporal gyrus (BA22, rostral) - Lt. middle |
| | temporal gyrus (BA37, dorsolateral) |
| #12 | Lt. precentral gyrus (BA4, trunk) - Lt. superior parietal |
| | lobule (BA7, rostral) |
| #13 | Lt. paracentral lobule (BA4, lower limb) - Lt. inferior |
| | temporal gyrus (BA37, ventrolateral) |
| #14 | Rt. posterior superior temporal sulcus (caudal) - Rt. |
| | inferior parietal lobule (BA39, rostrodorsal) |
| #15 | Rt. paracentral lobule (BA4, lower limb) - Rt. lingual |
| | gyrus (caudal) |
| #16 | Rt. inferior parietal lobule (BA40, rostroventral) - Lt. |
| | precuneus (BA7, medial) |
| #17 | Rt. superior temporal gyrus (BA41/42) - Lt. caudate |
| | (dorsal) |
| #18 | Rt. posterior superior temporal sulcus (caudal) - |
| | Hypothalamus |
| #19 | Lt. precentral gyrus (BA4, trunk) - Rt. superior parietal |
| | lobule (BA7, caudal) |
| #20 | Lt. inferior parietal lobule (BA39, rostrodorsal) - Lt. |
| | medial superior occipital gyrus |
| #21 | Lt. superior parietal lobule (BA7, postcentral) - Lt. |
| | inferior parietal lobule (BA39, caudal) |
| #22 | Rt. paracentral lobule (BA4, lower limb) - Rt. inferior |
| | occipital gyrus |
| #23 | Rt. precuneus (BA5, medial) - Lt. postcentral gyrus |
| | (BA1/2/3, trunk) |
| #24 | Rt. precuneus (BA5, medial) - Lt. VS/MT+ |
| #25 | Rt. superior temporal gyrus (BA41/42) - Lt. thalamus |
| | (caudal temporal) |
| #26 | Lt. middle frontal gyrus (BA6, ventrolateral) - Rt. |
| | precentral gyrus (BA4, upper limb) |
| #27 | Rt. superior parietal lobule (BA5, lateral) - Rt. inferior |
| | parietal lobule (BA40, rostroventral) |
| #28 | Lt. superior parietal lobule (BA5, intraparietal) - Rt. |
| | parietooccipital sulcus (ventromedial) |
| #29 | Rt. superior temporal gyrus (BA41/42) - Lt. thalamus |
| | (rostral temporal) |

TABLE 2

| Rank (# i) | Brain functional connectivity region |
|---|---|
| #30 | Rt. superior temporal gyrus (BA38, medial) - Rt. |
| | cerebellum (lobule VI) |
| #31 | Rt. superior temporal gyrus (BA38, medial) - Rt. |
| | lingual gyrus (rostral) |
| #32 | Rt. superior temporal gyrus (BA38, medial) - Rt. |
| | parahippocampal gyrus (area TL) |
| #33 | Lt. superior temporal gyrus (BA22, caudal) - Rt. |
| | superior temporal gyrus (BA22, rostral) |
| #34 | Lt. cerebellum (lobule IX) - Brainstem |
| #35 | Lt. parahippocampal gyrus (BA35/36, rostral) - |
| | Vermis. cerebellum (lobule IX) |
| #36 | Rt. superior temporal gyrus (BA38, medial) - Lt. |
| | inferior temporal gyrus (BA20, rostral) |
| #37 | Rt. superior temporal gyrus (BA38, medial) - Rt. |
| | parietooccipital sulcus (ventromedial) |
| #38 | Lt. precentral gyrus (BA4, head and face) - Lt. |
| | inferior temporal gyrus (BA20, rostral) |
| #39 | Lt. inferior temporal gyrus (BA20, rostral) - Lt. |
| | cingulate (BA23, caudal) |

The present invention is also directed to providing a method of providing information required for diagnosing sustained pain, which includes:

applying a stimulus to an individual and receiving brain image data according to the stimulus;

analyzing one or more selected from 1 to 39 brain functional connectivity listed in Tables 1 and 2; and

calculating a signature response based on the brain functional connectivity.

The present invention is also directed to providing a method for diagnosing sustained pain, which includes:

applying a stimulus to an individual and receiving brain image data according to the stimulus;

analyzing one or more selected from 1 to 39 brain functional connectivity listed in Tables 1 and 2; and

calculating a signature response based on the brain functional connectivity.

The present invention is also directed to providing a sustained pain diagnosis model which determines that a subject feels more sustained pain as the signature response calculated by Equation 1 below is higher.

Signature response= [symbol] · [symbol] =Σᵢ₌₁ⁿwᵢxᵢ. [Equation 1]

(Here, n is an integer of 1 to 39,

i is an integer of n or less,

wᵢ is a weight corresponding to the brain functional connectivity of #i, and

xᵢ is test data corresponding to the brain functional connectivity of #i.)

The present invention is also directed to providing a system for diagnosing sustained pain and determining the effect of relieving the sustained pain, comprising:

a receiver for receiving brain image data of a subject;

an analyzer for analyzing one or more selected from 1 to 39 brain functional connectivity regions listed in Tables 1 and 2 below from the received data; and

a calculator for calculating signature response based on the brain functional connectivity.

However, technical problems to be solved in the present invention are not limited to the above-described problems, and other problems which are not described herein will be fully understood by those of ordinary skill in the art from the following descriptions.

To achieve the above-described purposes, the present invention provides a biomarker composition for diagnosing sustained pain, which includes one or more selected from 1 to 39 brain functional connectivity regions listed in Tables 1 and 2 below.

TABLE 1

| Rank (# i) | Brain functional connectivity region |
|---|---|
| #1 | Lt. Inferior temporal gyrus (BA37, ventrolateral) - |
| | Lt. middle occipital gyrus |
| #2 | Lt. middle temporal gyrus (BA37, dorsolateral) - |
| | Lt. middle occipital gyrus |
| #3 | Lt. precentral gyrus (BA4, trunk) - Rt. precuneus |
| | (BA7, medial) |
| #4 | Lt. precuneus (BA7, medial) - Lt. postcentral gyrus |
| | (BA1/2/3, trunk) |
| #5 | Rt. inferior parietal lobule (BA40, rostrodorsal) - |
| | Lt. parietooccipital sulcus (dorsomedial) |
| #6 | Rt. precentral gyrus (BA4, upper limb) - Lt. inferior |
| | parietal lobule (BA39, rostrodorsal) |
| #7 | Lt. precentral gyrus (BA4, trunk) - Rt. medial superior |
| | occipital gyrus |
| #8 | Lt. paracentral lobule (BA4, lower limb) - Rt. |
| | precuneus (BA8, medial) |
| #9 | Lt. precentral gyrus (BA4, trunk) - Lt. precuneus (BA8, |
| | medial) |
| #10 | Rt. precuneus (BA7, medial) - Lt. postcentral gyrus |
| | (BA1/2/3, trunk) |
| #11 | Lt. superior temporal gyrus (BA22, rostral) - Lt. middle |
| | temporal gyrus (BA37, dorsolateral) |
| #12 | Lt. precentral gyrus (BA4, trunk) - Lt. superior parietal |
| | lobule (BA7, rostral) |
| #13 | Lt. paracentral lobule (BA4, lower limb) - Lt. inferior |
| | temporal gyrus (BA37, ventrolateral) |
| #14 | Rt. posterior superior temporal sulcus (caudal) - Rt. |
| | inferior parietal lobule (BA39, rostrodorsal) |
| #15 | Rt. paracentral lobule (BA4, lower limb) - Rt. lingual |
| | gyrus (caudal) |
| #16 | Rt. inferior parietal lobule (BA40, rostroventral) - Lt. |
| | precuneus (BA7, medial) |
| #17 | Rt. superior temporal gyrus (BA41/42) - Lt. caudate |
| | (dorsal) |
| #18 | Rt. posterior superior temporal sulcus (caudal) - |
| | Hypothalamus |
| #19 | Lt. precentral gyrus (BA4, trunk) - Rt. superior parietal |
| | lobule (BA7, caudal) |
| #20 | Lt. inferior parietal lobule (BA39, rostrodorsal) - Lt. |
| | medial superior occipital gyrus |
| #21 | Lt. superior parietal lobule (BA7, postcentral) - Lt. |
| | inferior parietal lobule (BA39, caudal) |
| #22 | Rt. paracentral lobule (BA4, lower limb) - Rt. inferior |
| | occipital gyrus |
| #23 | Rt. precuneus (BA5, medial) - Lt. postcentral gyrus |
| | (BA1/2/3, trunk) |
| #24 | Rt. precuneus (BA5, medial) - Lt. VS/MT+ |
| #25 | Rt. superior temporal gyrus (BA41/42) - Lt. thalamus |
| | (caudal temporal) |
| #26 | Lt. middle frontal gyrus (BA6, ventrolateral) - Rt. |
| | precentral gyrus (BA4, upper limb) |
| #27 | Rt. superior parietal lobule (BA5, lateral) - Rt. inferior |
| | parietal lobule (BA40, rostroventral) |
| #28 | Lt. superior parietal lobule (BA5, intraparietal) - Rt. |
| | parietooccipital sulcus (ventromedial) |
| #29 | Rt. superior temporal gyrus (BA41/42) - Lt. thalamus |
| | (rostral temporal) |

TABLE 2

| Rank (# i) | Brain functional connectivity region |
|---|---|
| #30 | Rt. superior temporal gyrus (BA38, medial) - Rt. |
| | cerebellum (lobule VI) |
| #31 | Rt. superior temporal gyrus (BA38, medial) - Rt. |
| | lingual gyrus (rostral) |
| #32 | Rt. superior temporal gyrus (BA38, medial) - Rt. |
| | parahippocampal gyrus (area TL) |
| #33 | Lt. superior temporal gyrus (BA22, caudal) - Rt. |
| | superior temporal gyrus (BA22, rostral) |
| #34 | Lt. cerebellum (lobule IX) - Brainstem |
| #35 | Lt. parahippocampal gyrus (BA35/36, rostral) - |
| | Vermis. cerebellum (lobule IX) |
| #36 | Rt. superior temporal gyrus (BA38, medial) - Lt. |
| | inferior temporal gyrus (BA20, rostral) |
| #37 | Rt. superior temporal gyrus (BA38, medial) - Rt. |
| | parietooccipital sulcus (ventromedial) |
| #38 | Lt. precentral gyrus (BA4, head and face) - Lt. |
| | inferior temporal gyrus (BA20, rostral) |
| #39 | Lt. inferior temporal gyrus (BA20, rostral) - Lt. |
| | cingulate (BA23, caudal) |

In addition, the present invention provides a system for diagnosing sustained pain, which includes:

a receiver for receiving brain image data of a subject;

an analyzer for analyzing one or more selected from 1 to 39 brain functional connectivity regions listed in Tables 1 and 2 below from the received data; and

a calculator for calculating a signature response based on the brain functional connectivity.

In one embodiment of the present invention, the brain image data may be magnetic resonance imaging data, but the present invention is not limited thereto.

In another embodiment of the present invention, the brain image data may be obtained by one selected from the group consisting of T1 magnetic resonance imaging (T1-MRI), T2-MRI, functional magnetic resonance imaging (fMRI), and resting-state functional magnetic resonance imaging (rsfMRI), but the present invention is not limited thereto.

In still another embodiment of the present invention, the signature response may be calculated by Equation 1 below, but the present invention is not limited thereto.

Signature response= [symbol] · [symbol] =Σᵢ₌₁ⁿwᵢxᵢ. [Equation 1]

(Here, n is an integer of 1 to 39,

i is an integer of n or less,

wᵢ is a weight corresponding to the brain functional connectivity of #i, and

xᵢ is test data corresponding to the brain functional connectivity of #i.)

In yet another embodiment of the present invention, the wᵢ is a weight corresponding to the brain functional connectivity of #i, listed in Tables 3 and 4, but the present invention is not limited thereto.

TABLE 3

| Rank | |
|---|---|
| (# i) | Weights |
| #1 | 0.0003308 |
| #2 | 0.0003259 |
| #3 | 0.0002891 |
| #4 | 0.0002799 |
| #5 | 0.0002773 |
| #6 | 0.0002771 |
| #7 | 0.0002635 |
| #8 | 0.0002634 |
| #9 | 0.0002541 |
| #10 | 0.0002474 |
| #11 | 0.0002400 |
| #12 | 0.0002322 |
| #13 | 0.0002285 |
| #14 | 0.0002265 |
| #15 | 0.0002182 |
| #16 | 0.0002085 |
| #17 | 0.0001976 |
| #18 | 0.0001834 |
| #19 | 0.0001829 |
| #20 | 0.0001754 |
| #21 | 0.0001744 |
| #22 | 0.0001726 |
| #23 | 0.0001654 |
| #24 | 0.0001625 |
| #25 | 0.0001584 |
| #26 | 0.0001518 |
| #27 | 0.0001333 |
| #28 | 0.0001313 |
| #29 | 0.0001304 |

TABLE 4

| Rank | |
|---|---|
| (# i) | Weights |
| #30 | −0.0004139 |
| #31 | −0.0003892 |
| #32 | −0.0003209 |
| #33 | −0.0003077 |
| #34 | −0.0003033 |
| #35 | −0.0003008 |
| #36 | −0.0002895 |
| #37 | −0.0002862 |
| #38 | −0.0002532 |
| #39 | −0.0001850 |

In yet another embodiment of the present invention, the sustained pain may be sustained over 10 seconds, but the present invention is not limited thereto.

In addition, the present invention provides a method of providing information required for diagnosing sustained pain, which includes:

applying a stimulus to an individual and receiving brain image data according to the stimulus;

analyzing one or more selected from 1 to 39 brain functional connectivity listed in Tables 1 and 2; and

calculating a signature response based on the brain functional connectivity.

In one embodiment of the present invention, the method may include determining that a subject feels pain the more the connectivity in Table 1 is present, but the present invention is not limited thereto.

In another embodiment of the present invention, the method may include determining that a subject feels pain the less the connectivity in Table 2 is present, but the present invention is not limited thereto.

In addition, the present invention provides a system for diagnosing sustained pain and determining the effect of relieving the sustained pain, comprising:

a receiver for receiving brain image data of a subject;

an analyzer for analyzing one or more selected from 1 to 39 brain functional connectivity regions listed in Tables 1 and 2 below from the received data; and

a calculator for calculating a signature response based on the brain functional connectivity.

In addition, the present invention provides a sustained pain diagnosis model which determines that a subject feels more sustained pain as the signature response calculated by Equation 1 below is higher:

Signature response= [symbol] · [symbol] =Σᵢ₌₁ⁿwᵢxᵢ. [Equation 1]

(Here, n is an integer of 1 to 39,

i is an integer of n or less,

wᵢ is a weight corresponding to the brain functional connectivity of #i, and xᵢ is test data corresponding to the brain functional connectivity of #i).

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIGS. 1A-1B show research questions and the overview of main analysis. FIG. 1A represents answering of three research questions (Q1-3) using several independent datasets (total of 6 studies, n=301) and a predictive modeling approach. FIG. 1B represents the overview of experiments and data analysis for answering the research questions. Participants acquired fMRI data while experiencing sustained pain induced by oral administration of capsaicin prior to fMRI scanning Many candidate models for predicting a pain rating based on a functional connectivity pattern during sustained pain experiences were generated (Study 1). The final model was selected through model competition using a pre-defined reference set based on predictive performance in learning and validation datasets (Studies 1 and 2). In addition, the final model for an additional dataset was validated (Studies 3 to 6). Different studies were used to answer different main research questions (that is, Q1 in Study 3, Q2 in Studies 4 and 5, and Q3 in Study 6).

FIG. 2A-2D are a diagrams visualizing the marker of sustained pain according to the present invention. Each section represents a regression weight between functional connectivity and the intensity of sustained pain between two brain regions. Red indicates a positive number, blue indicates a negative number, and the thickness and transparency of a line are proportional to a weight. Each part of the circle corresponds to a different brain region, the sum of positive weights of functional connectivity in each region is represented on the innermost layer, and the sum of negative weights thereof is represented on the middle layer. The outermost layer is colored to indicate which network is matched with each region according to classical brain network parcellation (Yeo et al., 2011, J Neurophysiol).

FIGS. 3A-3D are diagrams for detailed description on weight patterns of the marker of the present invention. FIG. 3A represents the marker of the present invention, in which columns and rows represent brain regions, and colors indicate weights of functional connectivity between two regions. The brain regions are grouped according to classical network parcellation. FIG. 3B represents an average of the weights of the marker of the present invention according to the group of brain regions (left), and the sum of only connections representing statistically significant weights according to a brain region group using bootstrap analysis (right). FIGS. 3C and 3D show the result of grouping “FIG. 3A” into anatomically similar regions and reconstructing the FIG. 3A (left), and the material that visualizes only statistically significant weights using the bootstrap analysis performed on this result (right).

FIGS. 4A-4D are a set of graphs illustrating prediction sensitivity/specificity of the marker of the present invention, corresponding to the result of testing the marker of the present invention with two datasets which are not used for learning of the marker of the present invention. By both datasets, the intensity of sustained pain felt by individuals were able to be significantly predicted. In the left graphs of FIG. 4A and FIG. 4C, the horizontal axis represents the intensity of actual pain, a vertical axis represents a predictive value, and each line is a regression line connecting data of different individuals. The color of each line indicates predictive power. In the right graphs of FIG. 4A and FIG. 4C, and gray represents actual pain, and red represents a predictive pain value. This marker did not respond to other sustained, aversive stimuli (a bitter taste stimulus and an odor stimulus), other than pain. FIG. 4B and FIG. 4D represent predictive values of the degree of aversiveness felt by an individual for each condition, and the case in which the value is high under a pain condition is shown in red, and the case in which the value is high under a aversive stimulus condition, rather than pain is shown in blue. For a statistical test, in FIG. 4A and FIG. 4C, bootstrap analysis was used, and in FIG. 4B and FIG. 4D, a t-test was used.

FIGS. 5A-5D are a set of graphs illustrating that a marker of the present invention has predictive power for clinical pain. FIG. 5A and FIG. 5B are test results for subacute or chronic back pain patients using the marker of the present invention, and although there is a difference according to experimental conditions, the present invention succeeded in predicting a difference in pain between individuals at a significant level was. The horizontal axis is actual pain, and the vertical axis is a predictive value. FIG. 5C and FIG. 5D are results showing that the marker of the present invention can successfully classify chronic back pain patients and a control. For a statistical test, a t-test was used.

FIGS. 6A-6B are diagrams for illustrating how the weight patterns of a marker of the present invention are in brain regions previously known to be related to pain. FIG. 6A shows weight patterns of the marker of the present invention in pain-associated brain regions such as prefrontal, somatosensory, subcortical and brainstem regions according to several bootstrap analysis-based thresholds. FIG. 6B shows the proportions of positive/negative weights of functional connections with a significance of P<0.05 from each region.

FIGS. 7A-7D are diagrams illustrating that a marker of the present invention is more similar to a model learned specifically for clinical pain than a model learned specifically for pain induced within a short time. FIG. 7A is the result of calculating the similarity between the marker of the present invention (model learned specifically for sustained pain) and other models (SBP model: subacute back pain model; EPP model: experimental phasic pain model) at a classical brain network parcellation level. Each colored circle corresponds to a different brain network. The marker of the present invention has a higher similarity to a subacute back pain model, compared with an EPP model. FIG. 7B and FIG. 7C are results showing network-level weight patterns and differences in weights of each marker and models based on bootstrap analysis. FIG. 7D shows differences in absolute values between a weight pattern of the marker of the present invention and weight patterns of other models. The marker of the present invention was more similar to the back pain model compared with the acute pain model in most networks (7 of 9 networks).

FIG. 8 is a set of graphs showing that a conventional marker (Neurologic Pain Signature, NPS) learned specifically for short-term pain is not suitable for predicting sustained pain. Two datasets used in this test are the same as those used in FIG. 4. In the left graphs, the horizontal axis represents the intensity of actual pain, the vertical axis represents a predictive value, and each line is a regression line connecting data of different individuals. The color of each line represents predictive power. In the right graphs, gray represents actual pain, and red represents a predictive pain value. For a statistical test, bootstrap analysis was used.

FIG. 9 is a set of schematic diagrams illustrating the present invention. The left diagram corresponds to a pain marker generation part, and the right diagram corresponds to a pain marker application part. In the pain marker generation part, functional magnetic resonance imaging (fMRI) is performed for 5 minutes or more while inducing sustained pain stimulation in a normal group without an underlying disease and clinical pain. Whole-brain functional connectivity is calculated using the acquired fMRI data, and a marker for predicting the intensity of pain is generated by modeling the relationship between functional connectivity and the intensity of pain reported by subjects during fMRI imaging through machine learning using principal component regression (PCR). In the pain marker application part, fMRI imaging is performed for 5 minutes or more after inducing sustained pain stimulation in a normal group not usually feeling pain, or maintaining a resting state without pain stimulation in a patient group experiencing pain, functional connectivity is calculated using the fMRI data, and then the intensity of pain is predicted using dot products obtained by comparing the pain marker obtained from the pain marker generation part with the functional connectivity.

FIG. 10 is a structural diagram of a system for diagnosing sustained pain and determining the effect of relieving the sustained pain according to one embodiment.

### DETAILED DESCRIPTION OF EXEMPLARY EMBODIMENTS

While terms used in the present invention have been selected from general terms currently used in a wide range where possible while considering functions in the present invention, this may vary depending on the intention of a person skill in the art, precedents, or the emergence of new technology. In addition, in specific cases, terms arbitrarily selected by the applicants may be used, and in this case, the meanings will be described in detail in the detailed description of the relevant invention. Therefore, the terms used herein should be defined based on the meanings of the terms, not simply the names thereof, and the content throughout the present invention.

Throughout the specification, when one part “includes” a component, it means that it may also include other components, not excluding components unless particularly stated otherwise. In addition, the term “˜ part” used herein refers to a unit of processing at least one function or operation, and may be implemented as hardware, software, or a combination of hardware and software.

If certain embodiments are otherwise implementable, specific steps may be performed in a different order from that described. For example, two steps described consecutively may be implemented substantially at the same time, or may be implemented in an opposite order to that described.

First, the present invention provides a biomarker composition for diagnosing sustained pain, which includes one or more selected from 1 to 39 brain functional connectivity regions listed in Tables 1 and 2 below.

TABLE 1

| Rank (# i) | Brain functional connectivity region |
|---|---|
| #1 | Lt. Inferior temporal gyrus (BA37, ventrolateral) - |
| | Lt. middle occipital gyrus |
| #2 | Lt. middle temporal gyrus (BA37, dorsolateral) - |
| | Lt. middle occipital gyrus |
| #3 | Lt. precentral gyrus (BA4, trunk) - Rt. precuneus |
| | (BA7, medial) |
| #4 | Lt. precuneus (BA7, medial) - Lt. postcentral gyrus |
| | (BA1/2/3, trunk) |
| #5 | Rt. inferior parietal lobule (BA40, rostrodorsal) - |
| | Lt. parietooccipital sulcus (dorsomedial) |
| #6 | Rt. precentral gyrus (BA4, upper limb) - Lt. inferior |
| | parietal lobule (BA39, rostrodorsal) |
| #7 | Lt. precentral gyrus (BA4, trunk) - Rt. medial superior |
| | occipital gyrus |
| #8 | Lt. paracentral lobule (BA4, lower limb) - Rt. |
| | precuneus (BA8, medial) |
| #9 | Lt. precentral gyrus (BA4, trunk) - Lt. precuneus (BA8, |
| | medial) |
| #10 | Rt. precuneus (BA7, medial) - Lt. postcentral gyrus |
| | (BA1/2/3, trunk) |
| #11 | Lt. superior temporal gyrus (BA22, rostral) - Lt. middle |
| | temporal gyrus (BA37, dorsolateral) |
| #12 | Lt. precentral gyrus (BA4, trunk) - Lt. superior parietal |
| | lobule (BA7, rostral) |
| #13 | Lt. paracentral lobule (BA4, lower limb) - Lt. inferior |
| | temporal gyrus (BA37, ventrolateral) |
| #14 | Rt. posterior superior temporal sulcus (caudal) - Rt. |
| | inferior parietal lobule (BA39, rostrodorsal) |
| #15 | Rt. paracentral lobule (BA4, lower limb) - Rt. lingual |
| | gyrus (caudal) |
| #16 | Rt. inferior parietal lobule (BA40, rostroventral) - Lt. |
| | precuneus (BA7, medial) |
| #17 | Rt. superior temporal gyrus (BA41/42) - Lt. caudate |
| | (dorsal) |
| #18 | Rt. posterior superior temporal sulcus (caudal) - |
| | Hypothalamus |
| #19 | Lt. precentral gyrus (BA4, trunk) - Rt. superior parietal |
| | lobule (BA7, caudal) |
| #20 | Lt. inferior parietal lobule (BA39, rostrodorsal) - Lt. |
| | medial superior occipital gyrus |
| #21 | Lt. superior parietal lobule (BA7, postcentral) - Lt. |
| | inferior parietal lobule (BA39, caudal) |
| #22 | Rt. paracentral lobule (BA4, lower limb) - Rt. inferior |
| | occipital gyrus |
| #23 | Rt. precuneus (BA5, medial) - Lt. postcentral gyrus |
| | (BA1/2/3, trunk) |
| #24 | Rt. precuneus (BA5, medial) - Lt. VS/MT+ |
| #25 | Rt. superior temporal gyrus (BA41/42) - Lt. thalamus |
| | (caudal temporal) |
| #26 | Lt. middle frontal gyrus (BA6, ventrolateral) - Rt. |
| | precentral gyrus (BA4, upper limb) |
| #27 | Rt. superior parietal lobule (BA5, lateral) - Rt. inferior |
| | parietal lobule (BA40, rostroventral) |
| #28 | Lt. superior parietal lobule (BA5, intraparietal) - Rt. |
| | parietooccipital sulcus (ventromedial) |
| #29 | Rt. superior temporal gyrus (BA41/42) - Lt. thalamus |
| | (rostral temporal) |

TABLE 2

| Rank (#i) | Brain functional connectivity region |
|---|---|
| #30 | Rt. superior temporal gyrus (BA38, medial) - Rt. |
| | cerebellum (lobule VI) |
| #31 | Rt. superior temporal gyrus (BA38, medial) - Rt. |
| | lingual gyrus (rostral) |
| #32 | Rt. superior temporal gyrus (BA38, medial) - Rt. |
| | parahippocampal gyrus (area TL) |
| #33 | Lt. superior temporal gyrus (BA22, caudal) - Rt. |
| | superior temporal gyrus (BA22, rostral) |
| #34 | Lt. cerebellum (lobule IX) - Brainstem |
| #35 | Lt. parahippocampal gyrus (BA35/36, rostral) - |
| | Vermis. cerebellum (lobule IX) |
| #36 | Rt. superior temporal gyrus (BA38, medial) - Lt. |
| | inferior temporal gyrus (BA20, rostral) |
| #37 | Rt. superior temporal gyrus (BA38, medial) - Rt. |
| | parietooccipital sulcus (ventromedial) |
| #38 | Lt. precentral gyrus (BA4, head and face) - Lt. |
| | inferior temporal gyrus (BA20, rostral) |
| #39 | Lt. inferior temporal gyrus (BA20, rostral) - Lt. |
| | cingulate (BA23, caudal) |

The term “marker” used herein shows brain functional connectivity, which is synchronized activity in anatomically distinguished brain regions.

In addition, the present invention provides a system for diagnosing sustained pain, which includes:

a receiver for receiving brain image data of a subject;

an analyzer for analyzing one or more selected from 1 to 39 brain functional connectivity regions listed in Tables 1 and 2 below from the received data; and

a calculator for calculating a signature response based on the brain functional connectivity.

The present invention also provides a system for diagnosing sustained pain and determining the effect of relieving the sustained pain, which includes:

a receiver for receiving brain image data of a subject;

an analyzer for analyzing one or more selected from 1 to 39 brain functional connectivity regions listed in Tables 1 and 2 below from the received data; and

a calculator for calculating a signature response based on the brain functional connectivity.

TABLE 1

| Rank (# i) | Brain functional connectivity region |
|---|---|
| #1 | Lt. Inferior temporal gyrus (BA37, ventrolateral) - |
| | Lt. middle occipital gyrus |
| #2 | Lt. middle temporal gyrus (BA37, dorsolateral) - |
| | Lt. middle occipital gyrus |
| #3 | Lt. precentral gyrus (BA4, trunk) - Rt. precuneus |
| | (BA7, medial) |
| #4 | Lt. precuneus (BA7, medial) - Lt. postcentral gyrus |
| | (BA1/2/3, trunk) |
| #5 | Rt. inferior parietal lobule (BA40, rostrodorsal) - |
| | Lt. parietooccipital sulcus (dorsomedial) |
| #6 | Rt. precentral gyrus (BA4, upper limb) - Lt. inferior |
| | parietal lobule (BA39, rostrodorsal) |
| #7 | Lt. precentral gyrus (BA4, trunk) - Rt. medial superior |
| | occipital gyrus |
| #8 | Lt. paracentral lobule (BA4, lower limb) - Rt. |
| | precuneus (BA8, medial) |
| #9 | Lt. precentral gyrus (BA4, trunk) - Lt. precuneus (BA8, |
| | medial) |
| #10 | Rt. precuneus (BA7, medial) - Lt. postcentral gyrus |
| | (BA1/2/3, trunk) |
| #11 | Lt. superior temporal gyrus (BA22, rostral) - Lt. middle |
| | temporal gyrus (BA37, dorsolateral) |
| #12 | Lt. precentral gyrus (BA4, trunk) - Lt. superior parietal |
| | lobule (BA7, rostral) |
| #13 | Lt. paracentral lobule (BA4, lower limb) - Lt. inferior |
| | temporal gyrus (BA37, ventrolateral) |
| #14 | Rt. posterior superior temporal sulcus (caudal) - Rt. |
| | inferior parietal lobule (BA39, rostrodorsal) |
| #15 | Rt. paracentral lobule (BA4, lower limb) - Rt. lingual |
| | gyrus (caudal) |
| #16 | Rt. inferior parietal lobule (BA40, rostroventral) - Lt. |
| | precuneus (BA7, medial) |
| #17 | Rt. superior temporal gyrus (BA41/42) - Lt. caudate |
| | (dorsal) |
| #18 | Rt. posterior superior temporal sulcus (caudal) - |
| | Hypothalamus |
| #19 | Lt. precentral gyrus (BA4, trunk) - Rt. superior parietal |
| | lobule (BA7, caudal) |
| #20 | Lt. inferior parietal lobule (BA39, rostrodorsal) - Lt. |
| | medial superior occipital gyrus |
| #21 | Lt. superior parietal lobule (BA7, postcentral) - Lt. |
| | inferior parietal lobule (BA39, caudal) |
| #22 | Rt. paracentral lobule (BA4, lower limb) - Rt. inferior |
| | occipital gyrus |
| #23 | Rt. precuneus (BA5, medial) - Lt. postcentral gyrus |
| | (BA1/2/3, trunk) |
| #24 | Rt. precuneus (BA5, medial) - Lt. VS/MT+ |
| #25 | Rt. superior temporal gyrus (BA41/42) - Lt. thalamus |
| | (caudal temporal) |
| #26 | Lt. middle frontal gyrus (BA6, ventrolateral) - Rt. |
| | precentral gyrus (BA4, upper limb) |
| #27 | Rt. superior parietal lobule (BA5, lateral) - Rt. inferior |
| | parietal lobule (BA40, rostroventral) |
| #28 | Lt. superior parietal lobule (BA5, intraparietal) - Rt. |
| | parietooccipital sulcus (ventromedial) |
| #29 | Rt. superior temporal gyrus (BA41/42) - Lt. thalamus |
| | (rostral temporal) |

TABLE 2

| Rank (# i) | Brain functional connectivity region |
|---|---|
| #30 | Rt. superior temporal gyrus (BA38, medial) - Rt. |
| | cerebellum (lobule VI) |
| #31 | Rt. superior temporal gyrus (BA38, medial) - Rt. |
| | lingual gyrus (rostral) |
| #32 | Rt. superior temporal gyrus (BA38, medial) - Rt. |
| | parahippocampal gyrus (area TL) |
| #33 | Lt. superior temporal gyrus (BA22, caudal) - Rt. |
| | superior temporal gyrus (BA22, rostral) |
| #34 | Lt. cerebellum (lobule IX) - Brainstem |
| #35 | Lt. parahippocampal gyrus (BA35/36, rostral) - |
| | Vermis. cerebellum (lobule IX) |
| #36 | Rt. superior temporal gyrus (BA38, medial) - Lt. |
| | inferior temporal gyrus (BA20, rostral) |
| #37 | Rt. superior temporal gyrus (BA38, medial) - Rt. |
| | parietooccipital sulcus (ventromedial) |
| #38 | Lt. precentral gyrus (BA4, head and face) - Lt. |
| | inferior temporal gyrus (BA20, rostral) |
| #39 | Lt. inferior temporal gyrus (BA20, rostral) - Lt. |
| | cingulate (BA23, caudal) |

The term “brain functional connectivity” used herein refers to synchronized activity shown in anatomically distinguished brain regions. That is, brain regions that are spatially separated but exhibit time-based similar activity patterns are functionally connected.

The present invention may include mapping a brain map by dividing the whole brain into specific regions (seeds) from brain image data of a subject. Specifically, the brain image data is obtained through magnetic resonance imaging (MRI) when the subject is relaxed while closing his/her eyes, and the MRI may be T1 magnetic resonance image (T1-MRI), T2-MRI or fMRI.

In addition, the fMRI may be obtained by further performing one or more of preprocessing selected from the group consisting of the realignment of artificial noise or noise caused by head movement, slice timing correction, spatial normalization, spatial smoothing and linear detrending.

The map may use one or more selected from the group consisting of the Harvard-Oxford atlas, the Brodmann's Atlas standard brain region map, Automated Anatomical Labeling (AAL), the Brainnetome Atlas, human connectome project-multi-modal parcellation (HCP-MMP), Buckner group parcellation, Stimulus Intensity Independent Signature-1 (SIIPS1), and Neurologic Pain Signature (NPS).

In addition, the brain map is mapped by analyzing values obtained from blood oxygenation level-dependent (BOLD) signal correlation coefficients between specific whole-brain regions, and the values obtained from BOLD signal correlation coefficients may be values obtained by quantifying brain functional connectivity, which may be test data of the present invention.

In the present invention, the signature response may be calculated by Equation 1 below, but the present invention is not limited thereto.

Signature response= [symbol] · [symbol] =Σᵢ₌₁ⁿwᵢxᵢ. [Equation 1]

(Here, n is an integer of 1 to 39,

i is an integer of n or less,

wᵢ is a weight corresponding to the brain functional connectivity of #i, and

xᵢ is test data corresponding to the brain functional connectivity of #i.)

In the present invention, the weight may be listed in Tables 3 and 4, but the present invention is not limited thereto.

TABLE 3

| Rank | |
|---|---|
| (#i) | Weights |
| #1 | 0.0003308 |
| #2 | 0.0003259 |
| #3 | 0.0002891 |
| #4 | 0.0002799 |
| #5 | 0.0002773 |
| #6 | 0.0002771 |
| #7 | 0.0002635 |
| #8 | 0.0002634 |
| #9 | 0.0002541 |
| #10 | 0.0002474 |
| #11 | 0.0002400 |
| #12 | 0.0002322 |
| #13 | 0.0002285 |
| #14 | 0.0002265 |
| #15 | 0.0002182 |
| #16 | 0.0002085 |
| #17 | 0.0001976 |
| #18 | 0.0001834 |
| #19 | 0.0001829 |
| #20 | 0.0001754 |
| #21 | 0.0001744 |
| #22 | 0.0001726 |
| #23 | 0.0001654 |
| #24 | 0.0001625 |
| #25 | 0.0001584 |
| #26 | 0.0001518 |
| #27 | 0.0001333 |
| #28 | 0.0001313 |
| #29 | 0.0001304 |

TABLE 4

| Rank | |
|---|---|
| (#i) | Weights |
| #30 | −0.0004139 |
| #31 | −0.0003892 |
| #32 | −0.0003209 |
| #33 | −0.0003077 |
| #34 | −0.0003033 |
| #35 | −0.0003008 |
| #36 | −0.0002895 |
| #37 | −0.0002862 |
| #38 | −0.0002532 |
| #39 | −0.0001850 |

In the present invention, the signature response is obtained by confirming one or the combination of two or more of the selected 39 connections of the present invention. According to the test data result in each connection of an individual, the signature response may be calculated by combining all of connection level predictive weights corresponding to the corresponding connections shown in Tables 3 and 4. For example, in Equation 1, when i is 1, and a test data value is 2, that is, when the test data value of [lower left temporal gyrus (BA37, ventrolateral)−left midoccipital gyrus] in an individual is 2, the signature response is increased by 0.0003308×2, and when the test data value of the corresponding connectivity marker is 1, the signature response is increased by 0.0003308×1. However, when the connection of [lower left temporal gyrus (BA37, ventrolateral)−left midoccipital gyrus] is not observed, that is, when the test data value is 0, the signature response is not increased. The signature response is obtained by identifying test data from any one or more connections of connections #1 to #39, and combining all the values obtained by multiplying the test data by assigned weights. As such, the sum of signature response derived from one or more connections is expressed as sigma (Σ). Since the signature response value has a different unit size depending on a machine acquiring the test data or method of calculating the test data, the absolute numerical standard of the intensity of pain expected according to the final combined signature response value. However, when two signature response values are generally compared, a higher signature response value than a small value means a higher level of sustained pain.

In the present invention, the test data value corresponds to a connectivity value extracted by analyzing the functional connectivity of specific whole-brain regions from brain image data. Specifically, from the brain image data, the whole brain is divided using one or more selected from the group consisting of Harvard-Oxford atlas, the Brodmann's Atlas standard brain region map, Automated the Anatomical Labeling (AAL), the Brainnetome Atlas, human connectome project-multi-modal parcellation (HCP-MMP), Buckner group parcellation, Stimulus Intensity Independent Signature-1 (SIIPS1), and Neurologic Pain Signature (NPS), and functional connectivity test data values in two specific regions may be extracted by calculating the correlation coefficient between a blood oxygen-level signal extracted from one region and a blood oxygen-level signal extracted from a divided region.

In the present invention, the sustained pain may be pain lasting for 10 seconds, 20 seconds, 30 seconds, 40 seconds, 50 seconds, 1 minute, 2 minutes, 3 minutes, 4 minutes, 5 minutes, 6 minutes, 7 minutes, 8 minutes, 9 minutes or 10 minutes or more, but the present invention is not limited thereto.

In the present invention, the sustained pain may be chronic pain, but the present invention is not limited thereto. The chronic pain refers to pain lasting over a common recovery period for an injury or disease. In one embodiment, the chronic pain is pain lasting longer than one week. The chronic pain may be persistent or intermittent. The general cause of the chronic pain may include, but not limited to, arthritis, cancer, reflex sympathetic dystrophy syndrome (RSDS), repetitive work-related stress disorders, herpes zoster, headaches, fibromyalgia, and diabetic neuropathy.

The “receiver” used herein receives brain image data from an external device. The brain image data may be image data indicating the degree of functional activity of the brain, for example, T1-MRI, T2-MRI, fMRI data, or rsfMRI data. The receiver sequentially collects brain image data according to time while a subject takes a rest with his/her eyes closed.

The “analyzer” used herein analyzes functional connectivity in a specific whole-brain region from brain image data, and extracts a connectivity value. Specifically, the whole brain is divided using one or more selected from the group consisting of the Harvard-Oxford atlas, the Brodmann's Atlas standard brain region map, Automated Anatomical Labeling (AAL), the Brainnetome Atlas, human connectome project-multi-modal parcellation (HCP-MMP), Buckner group parcellation, Stimulus Intensity Independent Signature-1 (SIIPS1), and Neurologic Pain Signature (NPS), and may extract functional connectivity values in two specific regions by calculating the correlation coefficient between a blood oxygen-level signal extracted from one region and a blood oxygen-level signal extracted from a divided region. In addition, in the analyzer, one or more functional connections may be displayed on a 2D image, and may be displayed separately from other connectivity. Here, predetermined dots may be displayed in each brain region, and connectivity of two or more regions may be displayed in a bar shape with a rainbow color.

The “calculator” used herein calculates a signature response based on the brain functional connectivity. Specifically, the calculator diagnoses a pain level of a subject by calculating a signature response using Equation 1.

The term “diagnosis” used herein encompasses determining the susceptibility of a subject to a particular disease or disorder, determining whether a subject currently has a particular disease or disorder, determining the prognosis of a subject with a particular disease or disorder, and therametrics (e.g., monitoring a subject state for providing information on treatment efficacy).

In addition, the present invention provides a method of providing information required for diagnosis of sustained pain, which includes:

applying a stimulus to an individual and receiving brain image data according to the stimulus;

analyzing one or more selected from 1 to 39 brain functional connectivity listed in Tables 1 and 2; and

calculating a signature response based on the brain functional connectivity.

In one embodiment of the present invention, the method may include determining that pain is felt the more the connectivity in Table 1 is present, but the present invention is not limited thereto.

In another embodiment of the present invention, the method may include determining that pain is felt the less the connectivity in Table 2 is present, but the present invention is not limited thereto.

The “stimulus” used herein may be an electrical stimulus, a visual stimulus, an auditory stimulus, or a taste stimulus, and preferably, a taste stimulus, but the present invention is not limited thereto. In one embodiment of the present invention, it was confirmed that the system of the present invention significantly distinguishes sustained stimuli caused by a spicy taste (e.g., capsaicin) among tastes by being compared with a stimulus caused by a bitter taste or odor.

In addition, the “stimulus” used herein may be an internal stimulus derived from a patient, rather than an external stimulus. In one embodiment of the present invention, it was confirmed that a patient with chronic pain caused by back pain and a normal person are significantly distinguished using the system of the present invention. As such, the stimulus of the present invention may be not only a stimulus derived from the outside, but also a sustained stimulus derived from the inside (e.g., a disease).

An individual (subject or individual) according to one embodiment of the present invention may include all or a part (e.g., the brain) of the body. While a part of the body with nerve activity is included without limitation, for example, the individual may include an organ such as the liver, heart, uterus, brain, breast and abdomen, but the present invention is not limited thereto. A part of the body may be or may not be separated from the individual, but the present invention is not limited thereto. Here, the individual may representatively be the human body, but is not limited to, and other animals (e.g., mammals such as a monkey, a mouse, a cow, a horse, a pig, a dog, sheep, a goat, a tiger, a rabbit, a snake, a chicken, a pig and a cat; mollusks such as squid, octopus, small octopus, webfoot octopus, clams, oysters and snails; and annelids such as earthworms, leeches and midges) may also be applicable.

Meanwhile, the embodiments of the present invention may be written with a program that can be executed on a computer, and may be implemented in a general-use digital computer which operates the program using a computer-readable recording medium.

Such a computer-readable recording medium may include a storage medium such as a magnetic storage medium (e.g., ROM, a floppy disk or a hard disk), an optical reading medium (e.g., CD-ROM or DVD) and a carrier wave (e.g., transmission over the internet).

In the present invention, the “receiver” applies a magnetic field and a high frequency to hydrogen atoms in body issue of a subject, and acquires MRI data from the subject in response thereto. That is, an image signal receiver is a part acquiring MRI data, and a device implemented with components already known in the field of magnetic resonance imaging such as a main magnetic field coil, a gradient coil, an RF coil and a magnetic room. Since the receiver may be a device well known to one of ordinary skill in the art, detailed description will be omitted.

The device according to the present invention may include a processor, a memory storing and running program data, a permanent storage part such as a disk drive, a communication port communicating with an external device, and an interface device such as a touch panel, keys or buttons. Methods implemented by a software module or algorithms may be stored on a computer-readable recording medium as computer-readable code or program instructions executable on the processor. Here, the computer-readable recording medium may be a magnetic storage medium (e.g., read-only memory (ROM), random-access memory (RAM), a floppy disk, or a hard disk), or an optical readable medium (e.g., CD-ROM or digital versatile disc (DVD)). The computer-readable recording medium may be distributed in networked computer systems, and may store and execute the computer-readable code in a distributed manner. The medium may be readable by a computer, stored in a memory, and executed on a processor.

This embodiment may be represented by functional block configurations and various processing steps. These functional blocks may be implemented with various numbers of hardware and/or software components, which implement specific functions. For example, the embodiment may employ integrated circuit components such as a memory, processor, logic and look-up table, capable of executing various functions by control of one or more microprocessors or other control devices. Similar to the components being executed with software programming or software elements, the embodiment may be implemented with a programing or scripting language such as C, C++, Java or Assembler by including various algorithms implemented with data structures, processors, routines or the combination of other programming components. Functional aspects may be implemented using algorithm(s) executed in one or more processors. In addition, the embodiment may employ the conventional art for electronic environment setting, signal processing, and/or data processing. The terms “mechanism,” “factor,” “means,” and “component” may be widely-used, mechanical and physical components, but the present invention is not limited thereto. The terms may include the meaning of a series of routines of software in association with a processor.

Specific runs which will be described in the specification are examples, and the technical range is not limited by any method. For simplicity of the specification, the description of conventional electronic components, control systems, software, and other functional aspects of the systems may be omitted. In addition, connections or connecting members of lines between components shown in the drawings are illustrative of functional connections and/or physical or circuit connections, and in an actual device, these connections may be represented as various alternative or additional functional connections, physical connections or circuit connections.

In the specification, when one component “includes” another component, this means that, unless specifically stated otherwise, other components may be further included, rather than excluded. The term “approximately” or “substantially” used herein are used at, or in the sense of proximity to, numerical values when manufacturing and material tolerances, which are inherent in the stated meanings, are provided. This term is used to prevent the unfair use of the disclosures in which correct or absolute values are cited to help in understanding the present invention by unscrupulous infringers.

Throughout the specification, the term “combination thereof” included in the Markush-type expression refers to a mixture or combination of one or more selected from the group consisting of constituents described in the Markush-type expression, that is, one or more selected from the group consisting of the components.

Hereinafter, to help in understanding the present invention, exemplary examples will be suggested. However, the following examples are merely provided to more easily understand the present invention, and not to limit the present invention.

### EXAMPLES

Experimental Materials and Methods

1. Summary

1-1. Experiment Summary

This study included eight independent fMRI studies (total N=448) to develop, validate and test a functional connection-based predictive model of sustained pain. A sample size was n=19 to 97 per study. Studies 1 to 3 and 6 and Supplementary Data 2 are datasets, which were collected for previous studies and not disclosed, and Studies 4 and 5 and Supplementary Data 1 are publicly available (OpenPain Project, available at http://www.openpain.org/). Study 1 played the role of a “learning dataset” and was used to develop and evaluate several candidate models. Study 2 was a “validation dataset” used only in evaluation of a candidate model. Studies 3 to 6 and Supplementary Data 1 and 2 are “independent test datasets” for testing and characterizing a final model in an unbiased manner. Studies 1 to 3 (n=109) and Supplementary Data 2 (n=58) are data sets obtained by inducing sustained pain in healthy participants using capsaicin. Studies 4 and 5 (n=192) and Supplementary Data 1 (n=56) were collected from subacute and chronic pain patients. Study 6 (n=33) shows data obtained by inducing phasic pain in healthy participants through delivery of thermal stimuli.

1-2. Brain Region Parcellation

In this experiment, four types of brain parcellation were used. First, Buckner group parcellation including the cerebral cortex, cerebellum and basal ganglia was combined with additional thalamic and brainstem regions provided in the SPM anatomy toolbox, and adjacent subregions in each network were divided into separate areas, thereby generating a total of 475 brain regions. Secondly, the Brainnetome Atlas was additionally combined with another brain parcellation including brainstem and cerebellum regions, thereby generating a total of 279 regions. Thirdly, Human Connectome Project multi-modal parcellation (HCP-MMP) was combined with subcortical regions of the Brainnetome Atlas and additionally with the brainstem and cerebellum regions, thereby generating a total of 249 regions. In the case of three types of parcellation, for additional functional connectivity, the BOLD signal time course was spatially averaged in each region. Finally, a total of 59 subregions which are known to be important in Neurologic Pain Signature (NPS) and Stimulus Intensity Independent Signature-1 (SIIPS1) models were used. In the case of the NPS and SIIPS1 subregions, the dot product between the data and the region prediction weight pattern was calculated, and a pattern expression value of each region was used. All brain parcellation regions were resampled in a voxel size of 3×3×3 mm³ and used.

2. Capsaicin Stimulation and Delivery Procedure (Studies 1 to 3)

In Studies 1 to 3, to minimize harm to participants and stably induce sustained pain, a spicy hot sauce (food ingredient) was applied to the tongue of each participant. In Studies 1 and 2, Tabasco® Habanero Pepper Sauce was used, and in Study 3, Capsaicin Hot Sauce (Jinmifoods, Inc.) was used. Specifically, a small amount, i.e., approximately 0.1 mL of hot sauce was dropped on a filter paper (2 cm*6.5 cm, rectangular-shaped), and then the hot sauce was spread on the top ⅓ part of the top surface of the filter paper in a round shape with a diameter of approximately 1 cm. While the participant was lying on a scanner, an experimenter handed a filter paper to the participant, and instructed the participant to carefully place the capsaicin-coated side of the filter paper on the tongue and close the mouth. After 30 seconds, after removing the filter paper, the participant was urged to open the mouth and breathe only through the mouth for 1 minute, so that the hot sauce applied to the tongue dried well and was allowed to sufficiently adhere capsaicin to the tongue. After 1 minute, simultaneously with the start of the scanning, the participants were asked to report their pain intensity on a screen using a trackball device while their mouths closed. Through the above-described stimulus delivery method, a method of 1) minimizing head movement which may occur during coughing in the scanner, 2) maximizing the intensity of pain while maintaining the pain within a tolerable range, and 3) delivering the pain may be easily made.

3. Bitter Taste Stimulation and Delivery Procedure (Studies 2 and 3)

To test the specificity of a sustained pain model, a bitter taste stimulus, which is a tongue stimulus which is not painful but is to avoided, was used in Studies 2 and 3. A small amount of quinine sulfate (50 mg) was dissolved in distilled water (0.1 ml), the corresponding quinine aqueous solution was transferred to a filter paper, and then a bitter taste to be avoided was induced in the same manner as the “Capsaicin stimulation and delivery procedures.”

4. Aversive Odor Stimulation and Delivery Procedure (Study 3)

As an additional aversive stimulus for testing the specificity of a sustained pain model, the smell of fermented skates was used. After attaching a piece of fermented skate covered with a filter paper to the inside of a mask designed to cover the mouth and nose of a participant, the participant was briefly pulled out of a scanner to allow him/her to wear a mask while breathing with the mouth. In addition, the participant was placed back into the scanner and instructed to continue breathing through the nose simultaneously with the start of scanning.

5. Capsaicin Delivery Using Gustometer System (Supplementary Data 2)

As another test for sustained pain, sustained pain was induced several times during scanning using a Gustometer system. The Gustometer system may deliver a fluid through an MR compatible mouthpiece, and to minimize an aversive sensation during scanning, the shape of the mouthpiece may be adjusted to fit the oral structure of the participant prior to the scanning. In Supplementary Data 2, the same type of hot sauce was used as in Study 3, but a hot sauce diluted in water (20 ml of the hot sauce, 80 ml of water) was used. The hot sauce dilution was delivered for 1.5 minutes at 1.5 minutes, 7 minutes after the start of the scanning During the scanning, to prevent the participant from swallowing the dilution, the delivered fluid was removed using a suction pump. The entire process of fluid delivery was controlled with OctaflowII (ALA Scientific Instruments Inc., Westbury, N.Y.), which is a computer-controlled 8-channel fluid delivery system.

6. Study 1: Capsaicin-Induced Sustained Pain Dataset (Learning Dataset)

In Study 1, 19 healthy right-handed participants were included (age=23.2±4.9 [mean±SD], 10 females). The participants were recruited from the Boulder/Denver Metro Areas, all of the participants voluntarily consented to participate in the experiment in written form, and participants with neurological/psychiatric disorders or corresponding contraindications, which make MRI scanning impossible, were excluded through an online questionnaire.

Two experimental conditions were experienced per participant. First, scanning was performed after sustained pain was induced by applying hot sauce under a capsaicin condition, and secondly, scanning was performed without pain stimulation under a control condition. Scanning according to each condition was continuously performed, and to minimize the amount of residual capsaicin which may remain on the tongue after hot sauce delivery, structural scans (T1 images) were taken between two scans. The order of capsaicin condition and control condition between the participants were counter-balanced.

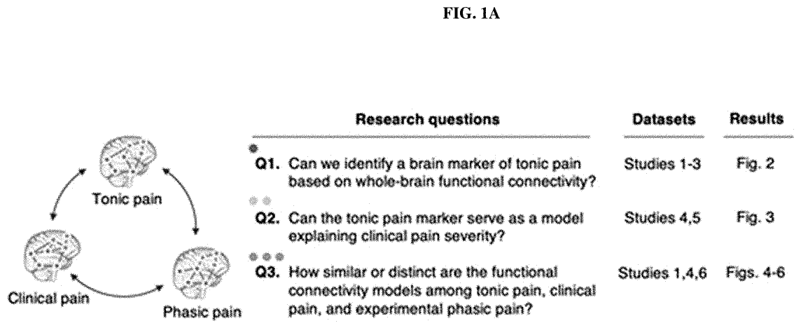

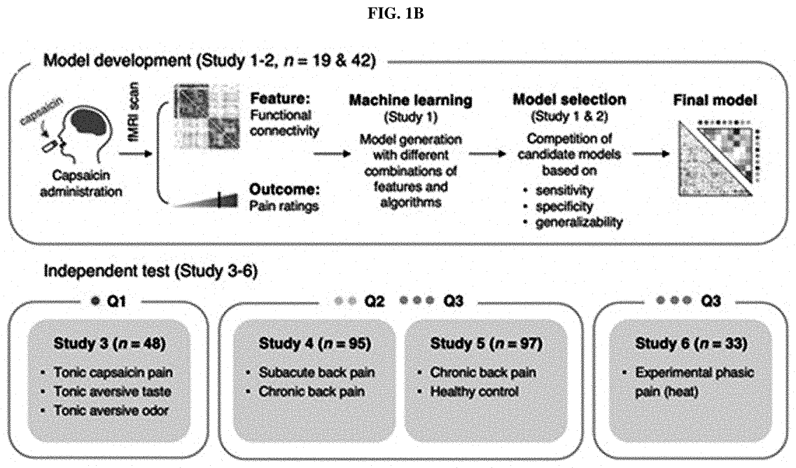

Scanning lasted for 5 minutes and 15 seconds per condition, and the participants evaluated the intensity and aversive sensation of sustained pain, which were felt by themselves every 45 seconds (a total of 7 times) from the start of scanning using an MR compatible trackball device. To minimize the effect generated by pain remaining after the capsaicin test, a liquid containing a small amount of sugar was provided after scanning. The experimental design for Study 1 is shown in FIG. 1B.

6-1. Rating Scale

The General Labeled Magnitude Scale (gLMS) was used as a pain rating scale. Anchors in gLMS start with “not at all (0)” on the far left of the scale, followed by “a little (0.061)”, “moderate (0.172)”, “strong (0.354)”, “very strong (0.533)”, and “strongest (the strongest imaginable sensation/aversive sensation of any type) (1)”.

6-2. fMRI Data Acquisition

Whole-brain fMRI data was acquired using a 3T Siemens TrioTim scanner of the University of Colorado Boulder. High-resolution T1-weighted structural images were acquired. EPI images were obtained with the following parameters (TR=460 ms, TE=29.0 ms, Multiband acceleration factor=8, FOV=248 mm, 82×82 matrix, spatial resolution=3×3×3 mm³, 56 interleaved slices, and volume number=685). Stimulus delivery and behavioral data acquisition were controlled using E-Prime software (PST Inc).

6-3. fMRI Data Analysis

Structural and functional MRI data was based on AFNI, FSL and SPMS, and preprocessed using an automated preprocessing pipe line developed by Mind Research Network (MRN). Parameters were acquired by co-registration of T1-weighted structural images to EPI images and normalization of T1 images to MNI images. After slice-timing correction and motion correction, the EPI images were normalized with a 3×3×3 mm³ MNI template using parameters normalizing T1 to MNI, followed by spatial smoothing with a 6-mm FWHM kernel. After automated preprocessing, for image intensity stabilization, 20 initial volumes of fMRI data were removed. Afterward, capsaicin and control conditions of different fMRI data were connected in one time series, and then the effects of (i) outliers in image intensity, (ii) a period of hand movement in relation to pain rating, and finally, (iii) nuisance variables related to 24 head movements (six head movement variables of x, y, z, roll, pitch and yaw and derivatives thereof, and the square of variables and the square of derivatives) were removed through regression analysis. The outliers were identified based on mean signal intensity, the Mahalanobis distance and the mean square of successive differences throughout volumes. After denoising through regression, winsorizing and a 0.1-Hz low pass filter were applied.

7. Study 2: Capsaicin-Induced Sustained Pain Dataset (Validation Dataset)

42 healthy right-handed participants were included except for 7 participants reporting higher avoidance rates under a control condition than the capsaicin condition. Other information is the same as that of Study 1.

For Study 2, there were three conditions: (1) capsaicin, (2) a bitter taste (quinin) and (3) a control condition. Capsaicin and bitter taste delivery procedures are shown in “Capsaicin stimulation and delivery procedures” and “Bitter taste stimulation and delivery procedures”. Scans for each of three conditions and structural image scans were performed consecutively, and the order was counter-balanced between participants. To obtain self-reports on common scales throughout various types of stimuli, avoidance rating scales were used instead of pain intensity or aversive sensations. The question was “How much do you want to avoid this experience in the future?”. Each scanning lasted 5 minutes and 10 seconds, and the participants provided avoidance ratings a total of 10 times every 30 seconds, starting from 10 seconds after the scanning had started. Other procedures were the same as in Study 1.

7-1. Rating Scale

As avoidance rating scales, the General Labeled Magnitude Scale (gLMS) was used. Anchors in gLMS start with “not at all (0)” on the far left of the scale, followed by “a little (0.061)”, “moderate (0.172)”, “strong (0.354)”, “very strong (0.533)”, and “strongest (the strongest imaginable sensation/aversive sensation of any type) (1)”.

7-2. fMRI Data Acquisition

Whole-brain fMRI data was acquired using a 3T Siemens TrioTim scanner of the University of Colorado Boulder. High-resolution T1-weighted structural images were acquired. EPI images were obtained with the following parameters (TR=460 ms, TE=27.2 ms, Multiband acceleration factor=8, FOV=220 mm, 82×82 matrix, spatial resolution=2.7×2.7×2.7 mm³, 56 interleaved slices, and volume number=676). Stimulus delivery and behavioral data acquisition were controlled using Matlab (Mathworks) and Psychtoolbox (http://psychtoolbox.org/).

The analysis of fMRI data was performed by preprocessing with the same pipe line as in Study 1.

8. Study 3: Capsaicin-Induced Sustained Pain Dataset (Independent Test Dataset)

Forty-eight healthy right-handed participants were included, except for four participants reporting higher avoidance rates under a control condition than a capsaicin condition and one participant not sufficient for including the whole brain in an MRI image. Participants were recruited from the Suwon area in Korea. Research was approved by the Institutional Review Committee of Sungkyunkwan University. Other information was the same as in Studies 1 and 2.

In Study 3, there were a total of four conditions: (i) capsaicin, (ii) a bitter taste (quinin), (iii) aversive odor (fermented skate), and (iv) a control condition. Capsaicin, bitter taste and aversive odor delivery procedures were described in the “Capsaicin stimulation and delivery procedure”, “Bitter taste stimulation and delivery procedure”, and “Aversive odor stimulation and delivery procedure”. Like Studies 1 and 2 described above, scanning was consecutively performed according to four experimental conditions, and the order was counter-balanced between the participants. Scanning according to each condition was continuously performed for 20 minutes, and the participants continuously reported avoidance rates during scanning using a trackball.

In this study, the experiment was designed to take images for a long time in order to sufficiently capture the increase and decrease in corresponding sensation per condition. In order to prevent the participants from falling asleep and maintaining a certain level of attention during scanning, at the moment when the avoidance rating marker changed from orange to red for a second every minute, the participants were instructed to click the left mouse button of the trackball. The other procedures were the same as in Study 2.

8-1. fMRI Data Acquisition

fMRI data was acquired in a 3T Siemens Prisma scanner of Sungkyunkwan University. Scanning parameters were the same as in Study 2, except that the volume number was 2608.

8-2. fMRI Data Analysis

Preprocessing was performed similarly to Studies 1 and 2, but there were also several differences. First, an automated MRN preprocessing pipe line was not used, and the same preprocessing steps were performed manually one by one. Secondly, regression analysis for removing the effect of a nuisance variable was performed after all scans were connected, but in this study, since one scan is sufficiently long, removal through regression was performed per scan. Thirdly, all of the time periods for mouse clicks to maintain attention were included as nuisance variables. Fourthly, 22 initial volumes instead of 20 were removed to ensure sufficient time for stabilizing image intensity.

9. Study 4: Clinical Back Pain Dataset (Acute and Chronic Back Pains)

Data in Study 4 was obtained from the OpenPain Project (OPP) database (http://www.openpain.org/). This dataset consisted of a longitudinal fMRI study for clinical back pain patients including 70 subacute back pain (SBP) patients (age=43.3±10.6 [mean±SD], 34 females) and 25 chronic back pain (CBP) patients (age=44.6±7.9 [mean±SD], 9 females).

All SBP patients included in this study showed an overall pain level higher than 40 based on a visual analogue scale (VAS; 0: no pain, 100: maximum imaginable pain), and the duration of back pain was 4 to 16 weeks. Patients had no pain symptoms in the past 12 months prior to the onset of their current pain symptoms. Participants with psychiatric, neurological or systemic disorders or high depression scores (BDI score: 19 points or more) were excluded.

9-1. fMRI Data Acquisition

Whole-brain fMRI data was acquired from a 3T Siemens TrioTim scanner. High-resolution T1-weighted structural images were acquired. EPI images were acquired with the following parameters (TR=2500 ms, TE=30 ms, 64×64 matrix, 3.4×3.4×3.0 mm³ spatial resolution, 36 interleaved slices, volume number=244).

9-2. fMRI Data Analysis

Resting-state fMRI data of the OPP database was preprocessed using a Fusion of Neuroimaging Preprocessing (FuNP) pipe line integrated with AFNI and FSL software. For image intensity stabilization, the first 4 volumes were removed. motion correction, slice timing correction and intensity normalization of 4D volume were applied. Head movement, white matter, cerebrospinal fluid, heart rate, arterial and vena cava-related nuisance variables were removed using ICA-based X-noiseifier (ICA-FIX) software (FMRIB). The fMRI data was normalized to the 3-mm³ MNI space after registration to the preprocessed T1 image. A 0.1-Hz low pass filter and 4-mm FWHM spatial smoothing were applied.

10. Study 5: Clinical Back Pain Dataset (Chronic Back Pain)

Like Study 5, the OPP database was used. This dataset included fMRI resting-state data of CBP patients and healthy elderly matched controls of two independent sites (Japan and UK). The Japan dataset consisted of 24 CBP patients (age=46.3±11.3 [mean±SD], 12 females) and 39 healthy control participants (age=39.1±13.5 [mean±SD], 14 females), and the UK dataset consisted of 17 CBP patients (age=44.0±11.4 [mean±SD], 12 females) and 17 healthy control participants (age=44.4±11.8 [mean±SD], 11 females). All CBP patients included in this study had pain symptoms for 12 months or more, and participants with psychiatric, neurological or systemic disorders and MRI contraindications were excluded.

Resting-state fMRI scanning was performed, and the participants kept their eyes open during scanning without any other tasks. Each run lasted for 9 minutes 45 seconds.

10-1. fMRI Data Acquisition

Whole-brain fMRI data was acquired in a 3T Siemens TrioTim scanner (CiNet (Osaka, Japan) or Addenbrooke Hospital (Cambridge, UK)). High-resolution T1-weighted structural images were acquired. In the case of the Japan dataset, EPI images were acquired with the following parameters (TR=2500 ms, TE=30 ms, FOV=212 mm, 64×64 matrix, 3.3×3.3×4.0 mm³ spatial resolution, 41 ascending slices, volume number=234).

In the case of the UK dataset, EPI images were acquired with the following parameters (TR=2000 ms, TE=30 ms, FOV=192 mm, 64×64 matrix, 3.0×3.0×3.8 mm³ spatial resolution, 32 interleaved slices, volume number=295). Data analysis was performed using the method described in Study 4.

11. Study 6: Heat-Induced Phasic Pain Dataset

33 healthy and right-handed participants were included (age=27.9±9.0 [mean±SD], 22 females). The participants were recruited in New York. Research had been approved by the Institutional Review Board of Columbia University, and all participants submitted written consent. The preliminary qualification for the participants was determined by an online questionnaire. Participants with psychiatric, neurological or systemic disorders and MRI contraindications were excluded.

Experimental phasic pain (EPP) was induced in the participants using thermal stimuli. The thermal stimuli were delivered to a left forearm surface. Each thermal stimulus lasted for 12.5 seconds, and consisted of 3 seconds of ramp-up, 7.5 seconds of plateau and 2 seconds of ramp-down. A total of 6 steps of temperatures (44.3±° C.-49.3±° C., 1° C. interval) were used for stimulation. After thermal stimulation, the participants reported ratings for (i) whether the stimulus was painful or not, and (ii) stimulus intensity.

The intensity for painless stimuli was defined as 0 to 100, and the intensity for painful stimuli was defined as 100 to 200. In this experiment, there were nine different runs, and 7 runs (1, 2, 4, 5, 6, 8 and 9) were designed for the participants to passively feel pain, and the other two runs (3 and 7) were designed for the participants to decrease or increase pain by themselves. In this study, only runs corresponding to passive pain experience data were used.

11-1. fMRI Data Acquisition

Whole-brain fMRI data was acquired in a 3T Philips Achieva TX scanner (University of Colorado Boulder). High-resolution T1-weighted structural images were acquired. EPI images were obtained with the following parameters (TR=2000 ms, TE=20 ms, parallel imaging, SENSE number=1.5, FOV=224 mm, 64×64 matrix, 3×3×3 mm³ spatial resolution, 42 interleaved slices, volume=213 (run corresponding to passive experience) or 195 (run corresponding to active experience)). Stimulus expression and behavioral data acquisition were controlled using E-Prime software (PST Inc).

11-2. fMRI Data Analysis

Preprocessing was performed using the same pipe line as used in the above-described methods. Structural T1-weighted images were co-registered to EPI images, followed by normalization with MNI. For image intensity stabilization, four fMRI data with initial volumes were removed. For such functional EPI images, slice-timing correction, motion-correction, and normalization to a MNI space were applied, followed by spatial smoothing with an 8-mm FWHM kernel. Afterward, data of a total of 9 runs for the preprocessed fMRI images were connected in one time series, and nuisance variables were regressed out.

Such nuisance variables include (i) an intercept corresponding to each run; (ii) a linear drift per run; (iii) 24 head movement variables; (iv) outlier timepoints; (v) indicator vectors for the first two images in each run; and (vi) white matter and cerebrospinal fluid signals. Afterward, a 1/180 Hz high pass filter was applied to the images.

### Example 1. Prediction of the Intensity of Capsaicin-Induced Sustained Pain

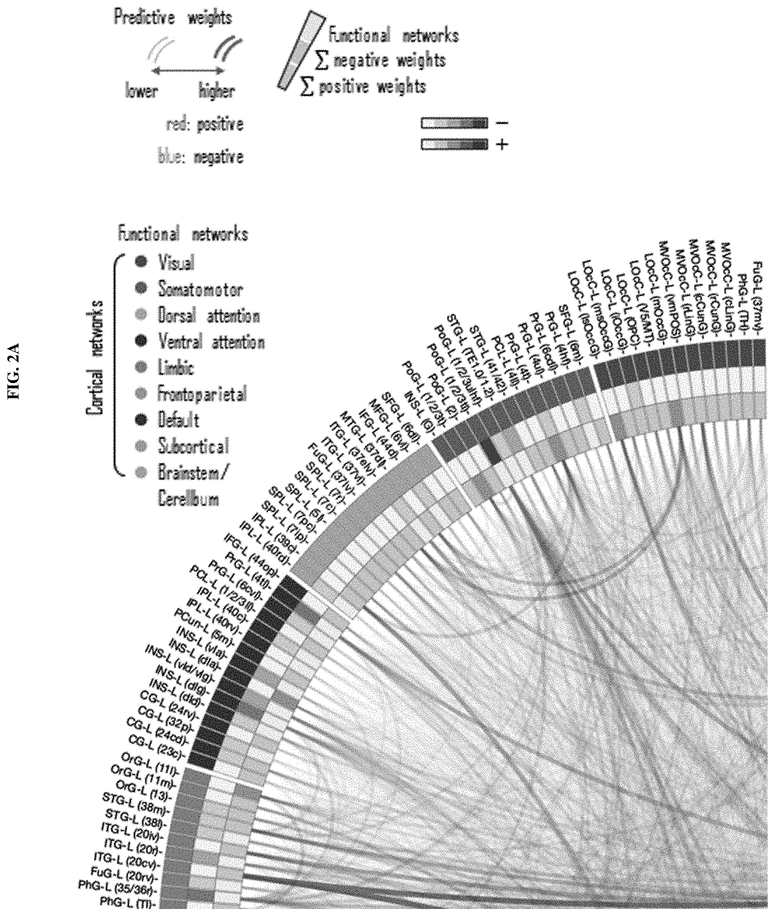

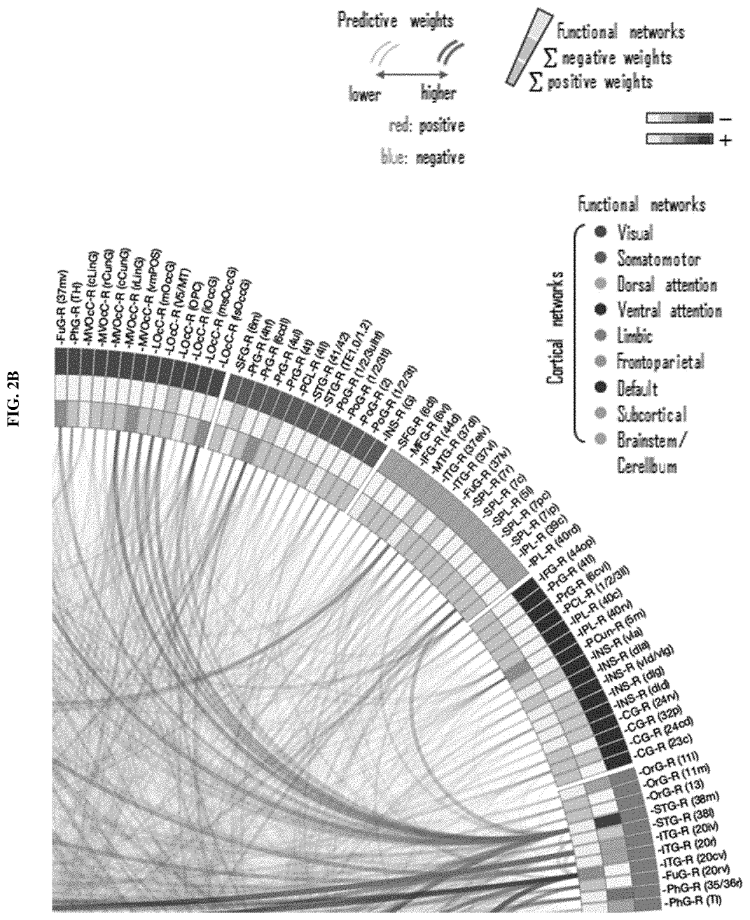

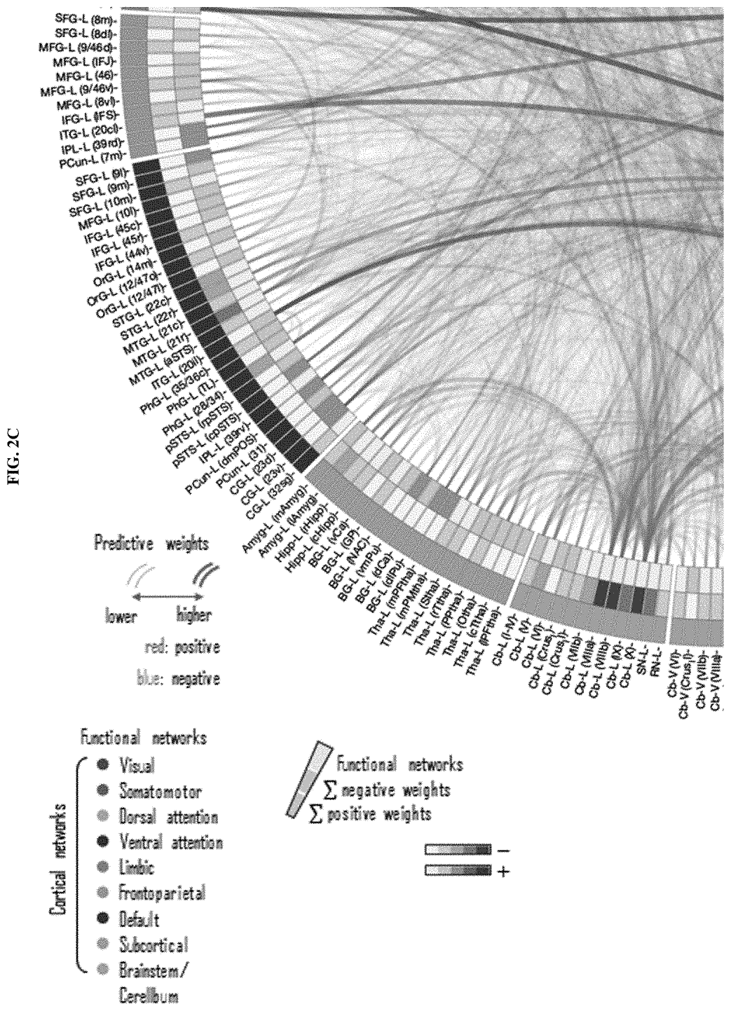

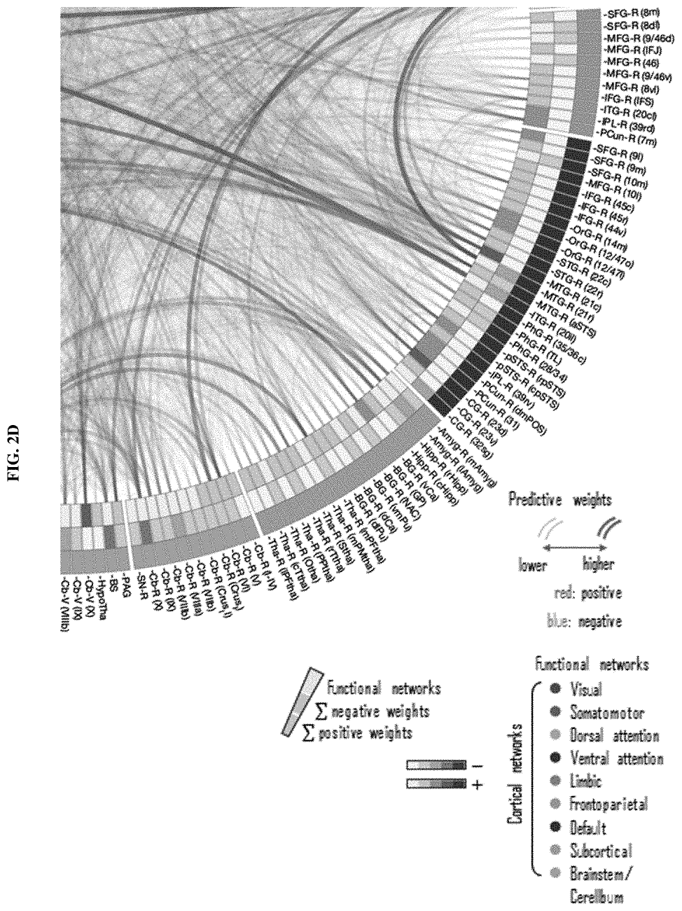

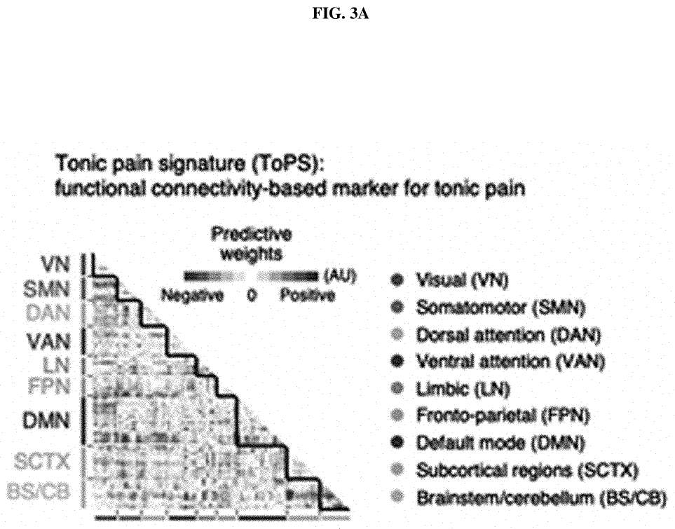

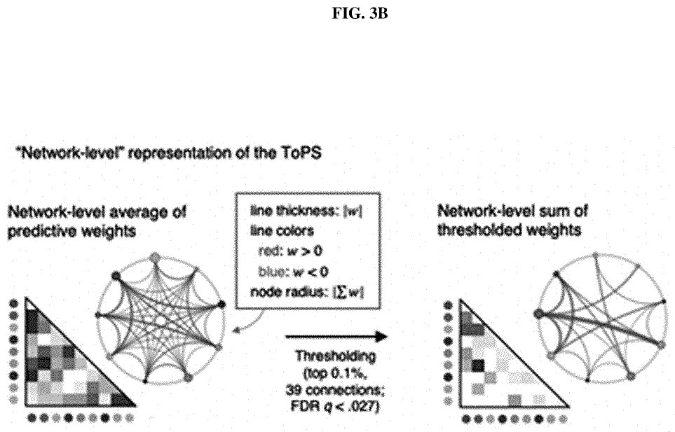

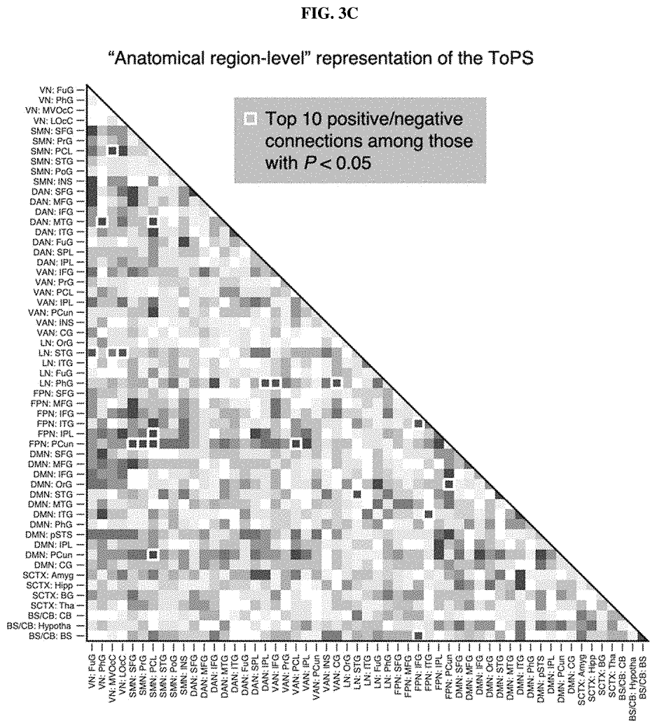

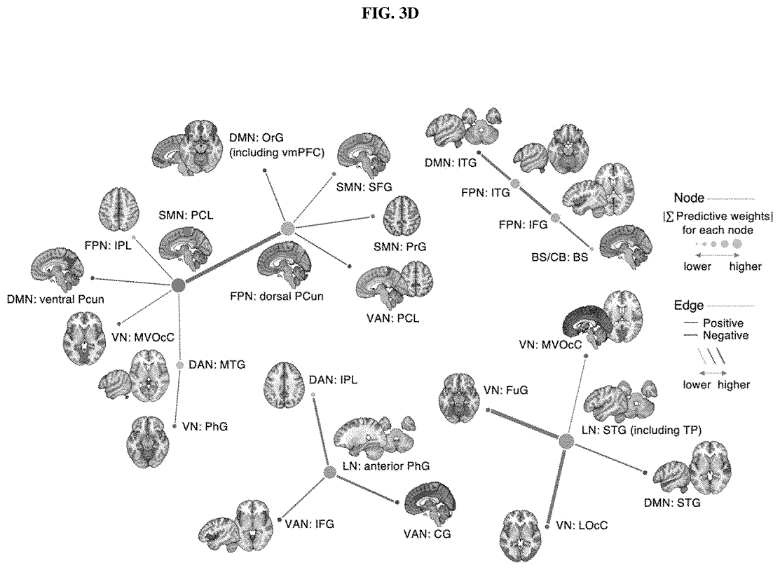

Markers obtained from a pain marker generation part are illustrated in FIGS. 2 and 3. Moreover, the top 39 markers are listed in Tables 5 and 6 below. The corresponding markers induced sustained pain in a total of 19 healthy subjects using capsaicin according to the above-described procedures, and the functional connectivity data at this time was modeled to predict the intensity of sustained pain. The functional connectivity data consisted of a total of 38,781 weight vectors.

TABLE 5

| | | | |
|---|---|---|---|
| | | | MNI |
| Rank | Weights | Regions | coordinates |

Positive connections

| | | | |
|---|---|---|---|
| #1 | −0.0003308 | Lt. inferior temporal gyrus (BA37, ventrolateral)-Lt. | (−58, −60, −6)- |
| | | middle occipital gyrus | (−34, −87, 13) |
| #2 | −0.0003259 | Lt. middle temporal gyrus (BA37, dorsolateral)-Lt. | (−61, −57, 7)- |
| | | middle occipital gyrus | (−34, −87, 13) |
| #3 | −0.0002891 | Lt. precentral gyrus (BA4, trunk)-Rt. precuneus | (−16, −21, 76)- |
| | | (BA7, medial) | (3, −63, 52) |
| #4 | −0.0002799 | Lt. precuneus (BA7, medial)-Lt. postcentral gyrus | (−7, −63, 52)- |
| | | (BA1/2/3, trunk) | (−22, −33, 70) |
| #5 | −0.0002773 | Rt. inferior parietal lobule (BA40, rostrodorsal)-Lt. | (45, −33, 46)- |
| | | parietoccipital sulcus (dorsomedial) | (−13, −66, 25) |
| #6 | −0.0002771 | Rt. precentral gyrus (BA4, upper limb)-Lt. inferior | (33, −21, 58)- |
| | | parietal lobule (BA39, rostrodorsal) | (−40, −60, 46) |
| #7 | −0.0002635 | Lt. precentral gyrus (BA4, trunk)-Rt. medial | (−16, −21, 76)- |
| | | superior occipital gyrus | (15, −64, 37) |
| #8 | −0.0002634 | Lt. paracentral lobule (BA4, lower limb)-Rt. | (−7, −21, 61)- |
| | | precuneus (BA7, medial) | (3, −63, 52) |
| #9 | −0.0002541 | Lt. precentral gyrus (BA4, trunk)-Lt. precuneus | (−16, −21, 76)- |
| | | (BA7, medial) | (−7, −63, 52) |
| #10 | −0.0002474 | Rt. precuneus (BA7, medial)-Lt. postcentral gyrus | (3, −63, 52)- |
| | | (BA1/2/3, trunk) | (−22, −33, 70) |
| #11 | −0.0002400 | Lt. superior temporal gyrus (BA22, rostral)-Lt. | (−58, −3, −9)- |
| | | middle temporal gyrus (BA37, dorsolateral) | (−61, −57, 7) |
| #12 | −0.0002322 | Lt. precentral gyrus (BA4, trunk)-Lt. superior | (−16, −21, 76)- |
| | | parietal lobule (BA7, rostral) | (−19, −60, 64) |
| #13 | −0.0002235 | Lt. paracentral lobule (BA4, lower limb)-Lt. inferior | (−7, −21, 61)- |
| | | temporal gyrus (BA37, ventrolateral) | (−58, −60, −6) |
| #14 | −0.0002265 | Rt. posterior superior temporal sulcus (caudal)-Rt. | (54, −39, 13)- |
| | | inferior parietal lobule (BA39, rostrodorsal) | (36, −63, 43) |
| #15 | −0.0002182 | Rt. paracentral lobule (BA4, lower limb)-Rt. lingual | (3, −21, 61)- |
| | | gyrus (caudal) | (9, −34, −6) |
| #16 | −0.0002085 | Rt. inferior parietal lobule (BA40, rostroventral)-Lt. | (54, −27, 28)- |
| | | precuneus (BA7, medial) | (−7, −63, 52) |
| #17 | −0.0001976 | Rt. superior temporal gyrus (BA41/42)-Lt. caudate | (51, −24, 13)- |
| | | (dorsal) | (−16, 4, 16) |
| #18 | −0.0001334 | Rt. posterior superior temporal sulcus (caudal)- | (54, −39, 13)- |
| | | Hypothalamus | (−1, 1, −9) |
| #19 | −0.0001829 | Lt. precentral gyrus (BA4. trunk)-Rt. superior | (−16, −21, 76)- |
| | | parietal lobule (BA7, caudal) | (15, −69, 55) |
| #20 | −0.0001754 | Lt. inferior parietal lobule (BA39, rostrodorsal)-Lt. | (−40, −60, 46)- |
| | | medial superior occipital gyrus | (−13, −87, 31) |
| #21 | −0.0001744 | Lt. superior parietal lobule (BA7, postcentral)-Lt. | (−25, −48, 67)- |
| | | inferior parietal lobule (BA39, caudal) | (−34, −78, 31) |
| #22 | −0.0001726 | Rt. paracentral lobule (BA4, lower limb)-Rt. inferior | (3, −21, 61)- |
| | | occipital gyrus | (30, −64, −9) |
| #23 | −0.0001654 | Rt. precuneus (BA5. medial)-Lt. postcentral gyrus | (6, −45, 58)- |
| | | (BA1/2/3, trunk) | (−22, −33, 70) |
| #24 | −0.0001625 | Rt. precuneus (BA7, medial)-Lt. V5/MT+ | (3, −63, 52)- |
| | | | (−49, −72, 7) |
| #25 | −0.0001584 | Rt. superior temporal gyrus (BA41/42)-Lt. thalamus | (51, −24, 13)- |
| | | (caudal temporal) | (−13, −21, 16) |
| #26 | −0.0001518 | Lt. middle frontal gyrus (BA6, ventrolateral)-Rt. | (−34, 4, 55)- |
| | | precentral gyrus (BA4, upper limb) | (33, −21, 58) |
| #27 | −0.0001333 | Rt. superior parietal lobule (BA5, lateral)-Rt. | (33, −42, 55)- |
| | | inferior parietal lobule (BA40, rostroventral) | (54, −27, 20) |
| #28 | −0.0001313 | Lt. superior parietal lobule (BA7, intraparietal)-Rt. | (−28, −57, 55)- |
| | | parietooccipital sulcus (ventromedial) | (12, −63, 13) |
| #29 | −0.0001304 | Rt. superior temporal gyrus (BA41/42)-Lt. thalamus | (51, −24, 13)- |
| | | (rostral temporal) | (−4, −15, 7) |

TABLE 6

| | | | |
|---|---|---|---|
| | | | MNI |
| Rank | Weights | Regions | coordinates |

Negative connections

| | | | |
|---|---|---|---|
| #30 | −0.0004139 | Rt. superior temporal gyrus (BA3 [symbol] , medial)-Rt. | (30, 16, −33)- |
| | | cerebellum (lobule VI) | (21, −54, −24) |
| #31 | −0.0003892 | Rt. superior temporal gyrus (BA33, medial)-Rt. | (30, 16, −33)- |
| | | lingual gyrus (rostral) | (15, −57, −6) |
| #32 | −0.0003209 | Rt. superior temporal gyrus (BA3 [symbol] , medial)- | (30, 16, −33)- |
| | | Rt. parahippocampal gyrus (area TL) | (27, −30, −15) |
| #33 | −0.0003077 | Lt. superior temporal gyrus (BA22, caudal)-Rt. | (−64, −33, 7)- |
| | | superior temporal gyrus (BA22, rostral) | (54, −12, −3) |
| #34 | −0.0003033 | Lt. cerebellum (lobule IX)-Brainstem | (−10, −51, −42)- |
| | | | (−1, −24, −27) |
| #35 | −0.0003008 | Lt. parahippocampal gyrus (BA35/36, rostral)- | (−31, −6, −33)- |
| | | Vermis, cerebellum (lobule IX) | (−4, −54, −36) |
| #36 | −0.0002895 | Rt. superior temporal gyrus (BA38, lateral)-Lt. | (45, 13, −13)- |
| | | inferior temporal gyrus (BA20, rostral) | (−46, −3, −39) |
| #37 | −0.0002862 | Rt. superior temporal gyrus (BA38, medial)-Rt. | (30, 16, −33)- |
| | | parietooccipital sulcus (ventromedial) | (12, −63, 13) |
| #38 | −0.0002532 | Rt. precentral gyrus (BA4, head and face)- | (51, −3, 34)- |
| | | Lt. inferior temporal gyrus (BA20, rostral) | (−46, −3, −39) |
| #39 | −0.0001850 | Lt. inferior temporal gyrus (BA20, rostral)-Lt. | (−46, −3, −39)- |
| | | cingulate gyrus (BA23, caudal) | (10, −24, 43) |

[symbol]

indicates data missing or illegible when filed

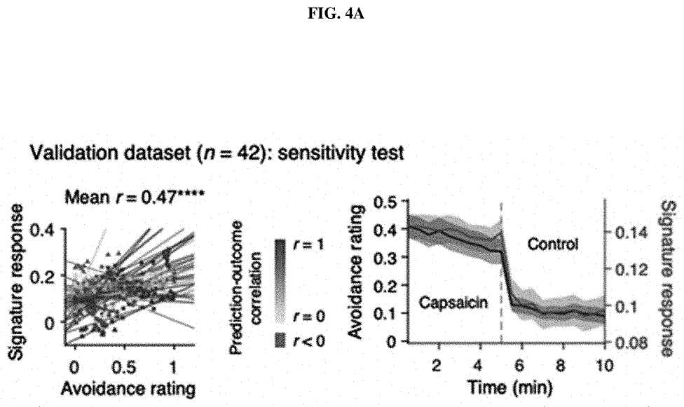

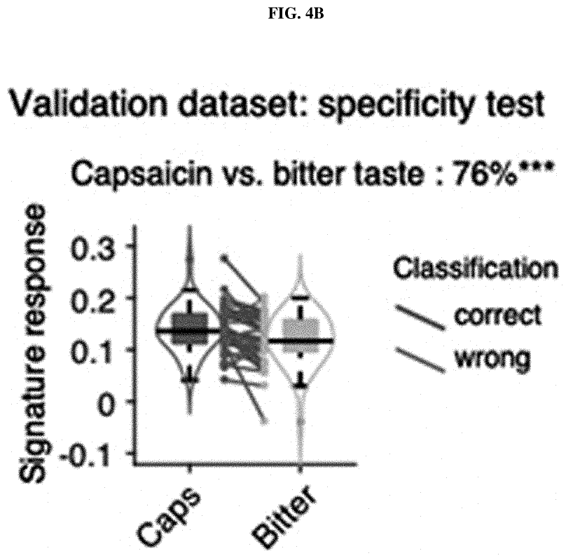

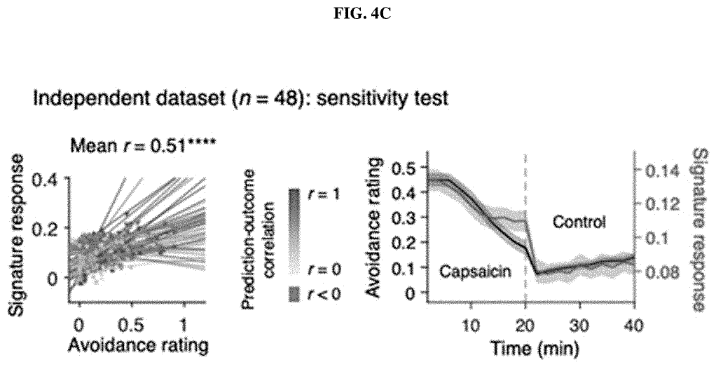

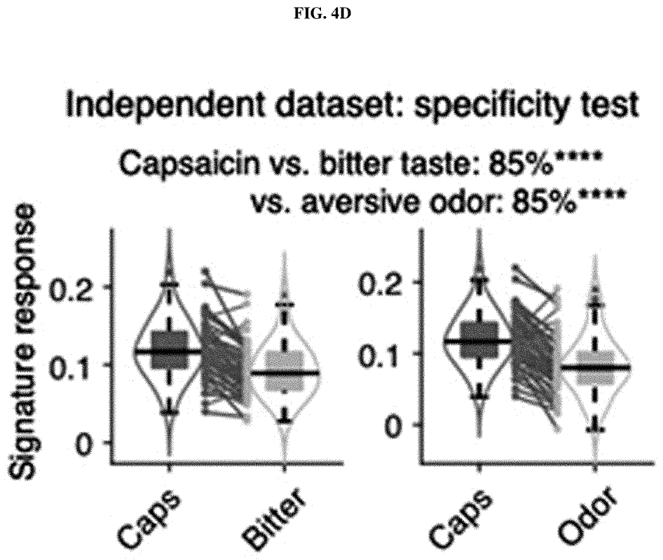

In the pain marker application part, predictive examples of sustained pain in a normal group are shown in FIG. 4 using the markers shown in FIGS. 2 and 3. From two datasets which were not used to generate the corresponding markers (Study 2: 42, Study 3: 48), the intensity of capsaicin-induced sustained pain reported to be intermittent by the subjects was predicted to be very significant (r=0.47-0.51), and particularly, one (Study 3) of the two datasets is a dataset which is not used to train a model or select a final model, and shows robustness of the corresponding markers.

In addition, as shown in FIG. 4, the corresponding markers are specific for pain, and do not respond to other aversive stimuli (pain vs. bitter taste identification test: 76-85%; pain vs. aversive odor identification test: 85%).

### Example 2. Prediction of Intensity of Sustained Pain Caused by Back Pain

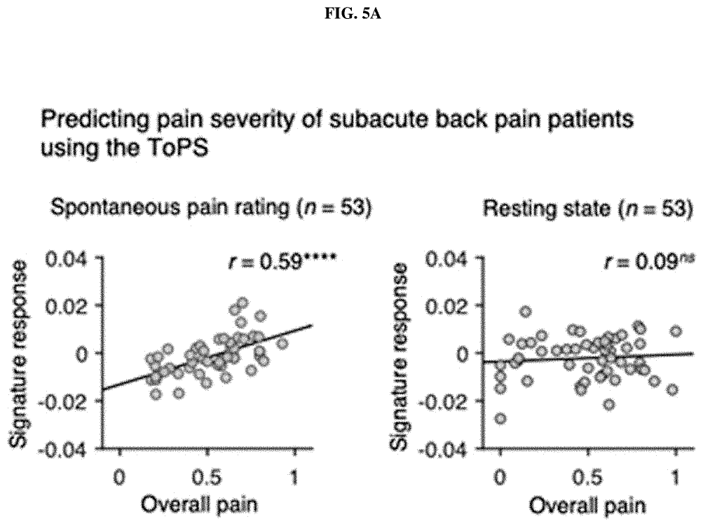

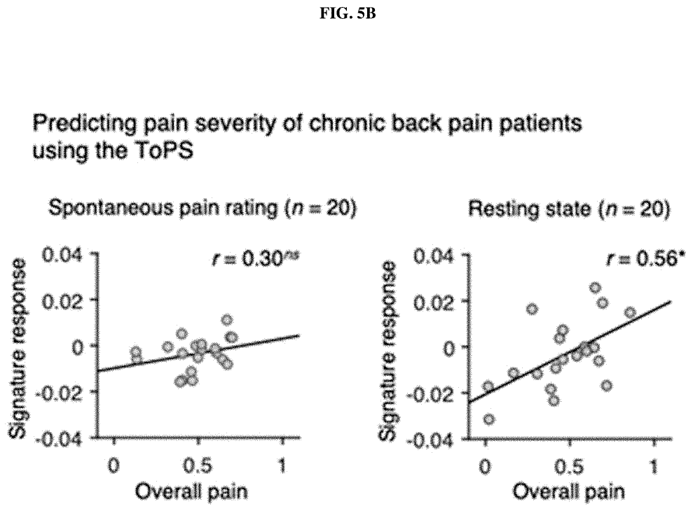

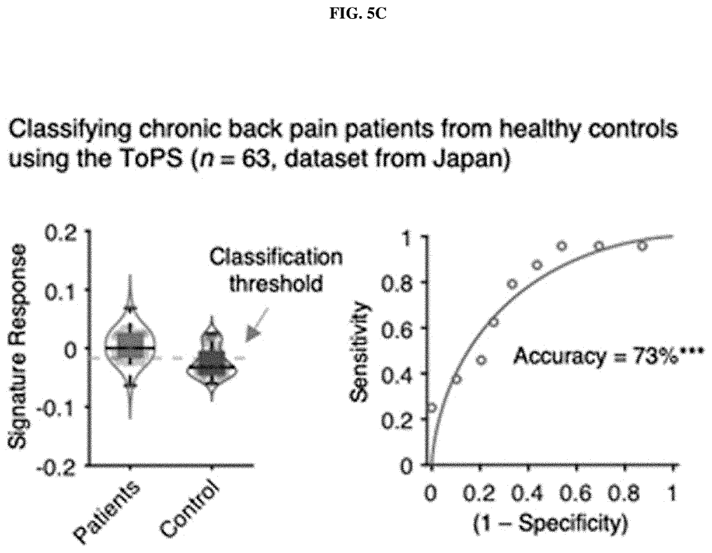

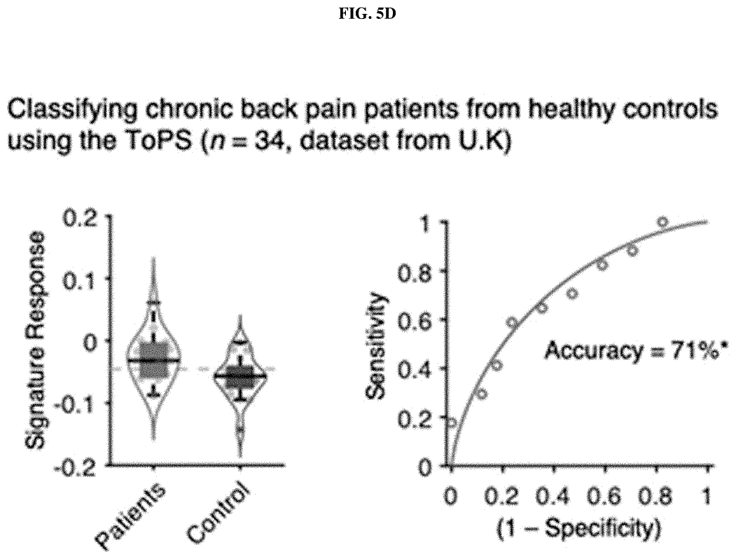

In the pain marker application part, examples of predicting the overall pain intensity of a clinical back pain patient group using the markers shown in FIGS. 2 and 3 are shown in FIG. 5. From the datasets for subacute back pain (53 patients) and chronic back pain (20 patients) patient groups, which were not used to generate the corresponding markers, the corresponding markers showed significant predictive power (subacute: r=0.57; chronic: r=0.56), and from the other two datasets consisting of a chronic back pain patient group (63 patients) and an age-matched normal control group (34 patients), the patient group and the control group were classified with significant accuracy (73%, 71%).

This shows that the present invention can be used to diagnose chronic pain patients and monitor their pain intensity, which are considered clinically important, by an objective and accurate method.

### Example 3. Comparison of Predictive Effect with Conventional Pain Prediction Model

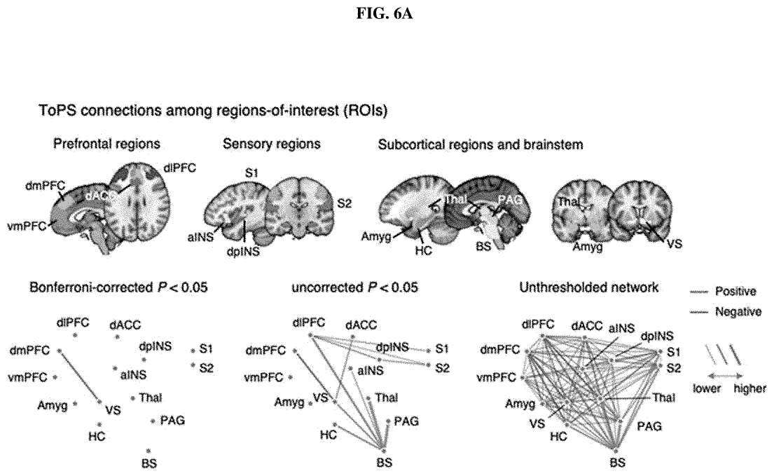

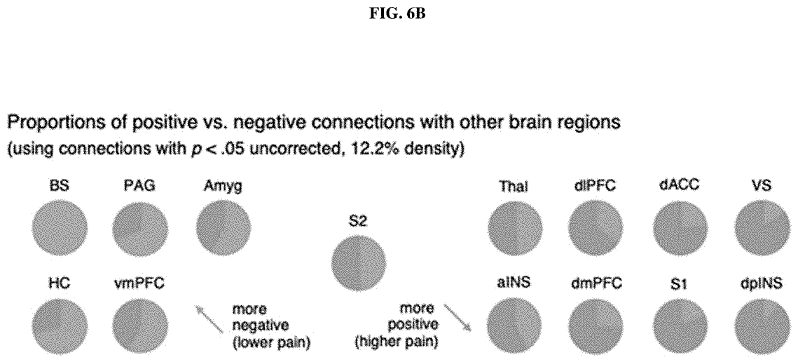

The results of confirming the weight pattern of the markers defined in the present invention in brain regions well known to be related to pain are shown in FIG. 6. Based on the fact that the markers of the present invention predicted sustained pain to a significant extent, the weight pattern information shown in FIG. 6 shows the possibility of contributing to the development of treatment methods such as brain stimulation by providing information on whether pain will increase or decrease when the corresponding region is stimulated.

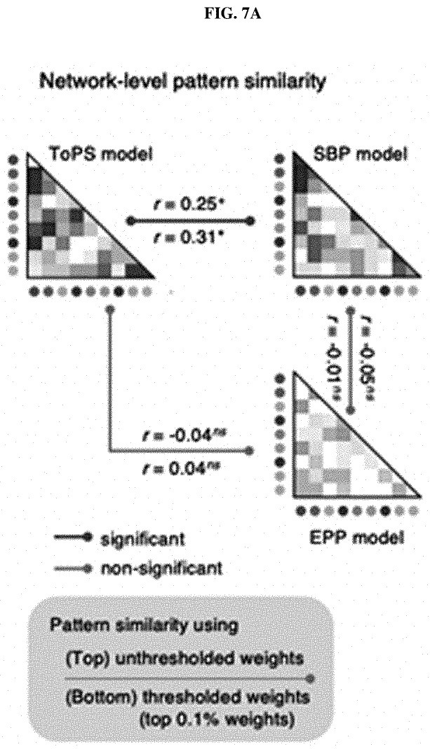

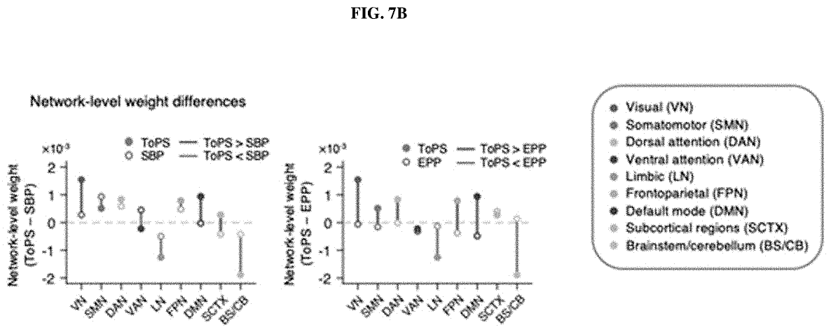

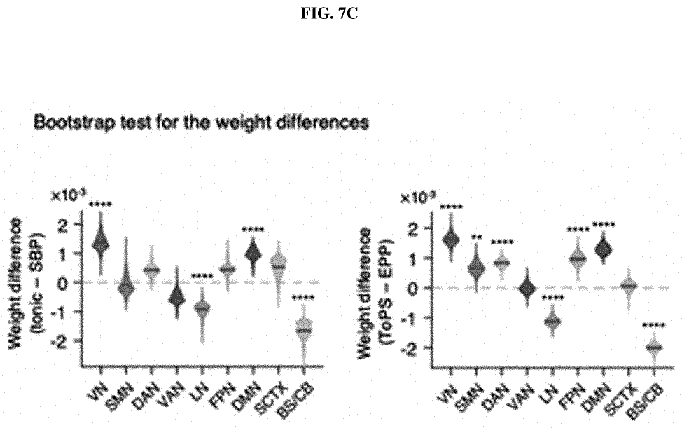

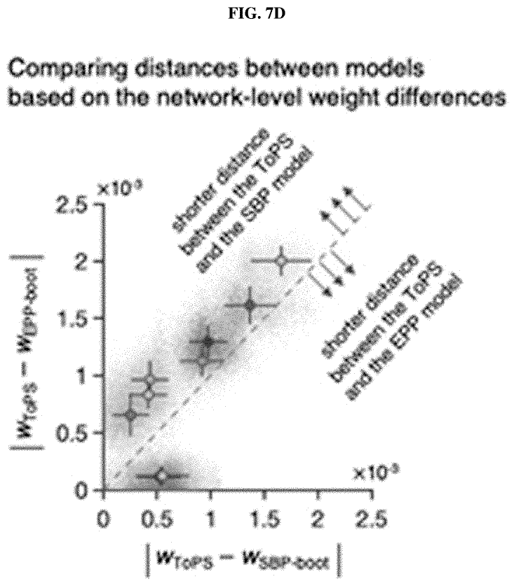

FIG. 7 shows that the weight pattern of the marker defined in the present invention is more similar to a model (subacute back pain model) specifically trained for overall pain of a patient group suffering from back pain, compared to a model (acute pain model) specifically trained for pain induced for a very short time in healthy patients. This shows that sustained pain is more likely to be clinically applied than acute pain.

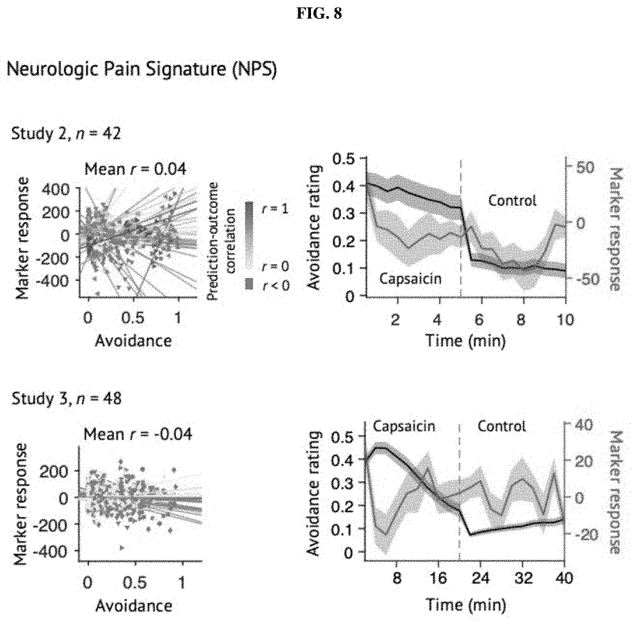

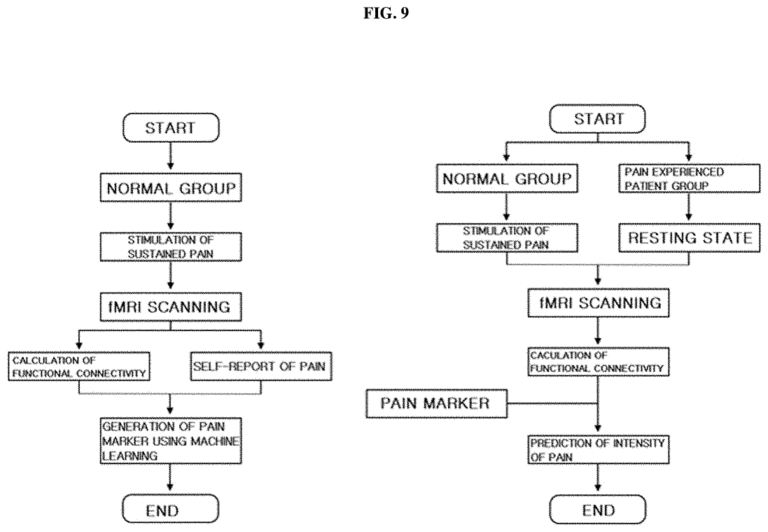

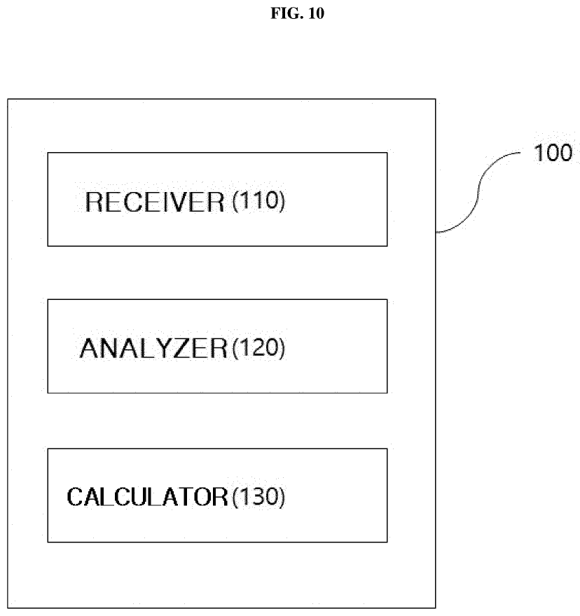

FIG. 8 shows the result of using the Neurologic Pain Signature (NPS) of a marker specific for pain induced for a very short time, disclosed in 2016, to predict sustained pain.

As shown in FIG. 8, NPS was not successful in predicting sustained pain, demonstrating that the marker defined in the present invention has a superior effect in predicting sustained pain, compared to the conventional marker.

The marker of the present invention is specific for pain, and does not respond to other noxious stimuli. This shows that the present invention can be used for monitoring the intensity of pain and a response to treatment of chronic pain patients, which are considered clinically significant, by an objective and precise method. In addition, by comparing a responsive clinical pain group and a non-responsive clinical pain group in the present invention, there is also the possibility in which the present invention can be used for differential diagnosis for the cause of pain. In addition, the present invention can be used for pre-screening of a drug clinical trial to dramatically reduce time and costs consumed in the trial, and can contribute to the development of pain treatment methods such as brain stimulation based on a weight pattern of the marker. Finally, the present invention is expected to be used to measure the intensity of pain in groups which have difficulty in reporting pain (a vegetative state, aphasia patients, the elderly, infants, etc).

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

### EXPLANATION OF REFERENCE NUMERALS AND MARKS

100: system for diagnosing sustained pain
110: receiver
120: analyzer
130: calculator
AU: arbitrary unit
Amyg: amygdala
BS: brainstem
BG: basal ganglia
CB: cerebellum
CG: cingulate gyrus
FuG: fusiform gyrus
Hipp: hippocampus
Hypotha: hypothalamus
IFG: inferior frontal gyrus
INS: insular gyrus
IPL: inferior parietal lobule
ITG: inferior temporal gyrus
LOcC: lateral occipital cortex
MFG: middle frontal gyrus
MTG: middle temporal gyrus
MVOcC: medioventral occipital cortex
OrG: orbital gyrus
PCL: paracentral lobule
PCun: precuneus
PhG: parahippocampal gyrus
PoG: postcentral gyrus
PrG: precentral gyrus
pSTS: posterior superior temporal sulcus
SFG: superior frontal gyrus
SPL: superior parietal lobule
STG: superior temporal gyrus
Tha: thalamus

What is claimed is:

1. A biomarker composition for predicting sustained pain, comprising:
one or more of 1 to 39 brain functional connectivity regions, which are listed in Table 1 and 2 below.
TABLE 1

| Rank (#i) | Brain functional connectivity region |
|---|---|
| #1 | Lt. Inferior temporal gyrus (BA37, ventrolateral)-Lt. middle occipital gyrus |
| #2 | Lt. middle temporal gyrus (BA37, dorsolateral)-Lt. middle occipital gyrus |
| #3 | Lt. precentral gyrus (BA4, trunk)-Rt. precuneus (BA7, medial) |
| #4 | Lt. precuneus (BA7, medial)-Lt. postcentral gyrus (BA1/2/3, trunk) |
| #5 | Rt. inferior parietal lobule (BA40, rostrodorsal)-Lt. parietooccipital sulcus |
| | (dorsomedial) |
| #6 | Rt. precentral gyrus (BA4, upper limb)-Lt. inferior parietal lobule (BA39, |
| | rostrodorsal) |
| #7 | Lt. precentral gyrus (BA4, trunk)-Rt. medial superior occipital gyrus |
| #8 | Lt. paracentral lobule (BA4, lower limb)-Rt. precuneus (BA8, medial) |
| #9 | Lt. precentral gyrus (BA4, trunk)-Lt. precuneus (BA8, medial) |
| #10 | Rt. precuneus (BA7, medial)-Lt. postcentral gyrus (BA1/2/3, trunk) |
| #11 | Lt. superior temporal gyrus (BA22, rostral)-Lt. middle temporal gyrus (BA37, |
| | dorsolateral) |
| #12 | Lt. precentral gyrus (BA4, trunk)-Lt. superior parietal lobule (BA7, rostral) |
| #13 | Lt. paracentral lobule (BA4, lower limb)-Lt. inferior temporal gyrus (BA37, |
| | ventrolateral) |
| #14 | Rt. posterior superior temporal sulcus (caudal)-Rt. inferior parietal lobule (BA39, |
| | rostrodorsal) |
| #15 | Rt. paracentral lobule (BA4, lower limb)-Rt. lingual gyrus (caudal) |
| #16 | Rt. inferior parietal lobule (BA40, rostroventral)-Lt. precuneus (BA7, medial) |
| #17 | Rt. superior temporal gyrus (BA41/42)-Lt. caudate (dorsal) |
| #18 | Rt. posterior superior temporal sulcus (caudal)-Hypothalamus |
| #19 | Lt. precentral gyrus (BA4, trunk)-Rt. superior parietal lobule (BA7, caudal) |
| #20 | Lt. inferior parietal lobule (BA39, rostrodorsal)-Lt. medial superior occipital gyrus |
| #21 | Lt. superior parietal lobule (BA7, postcentral)-Lt. inferior parietal lobule (BA39, |
| | caudal) |
| #22 | Rt. paracentral lobule (BA4, lower limb)-Rt. inferior occipital gyrus |
| #23 | Rt. precuneus (BA5, medial)-Lt. postcentral gyrus (BA1/2/3, trunk) |
| #24 | Rt. precuneus (BA5, medial)-Lt. VS/MT+ |
| #25 | Rt. superior temporal gyrus (BA41/42)-Lt. thalamus (caudal temporal) |
| #26 | Lt. middle frontal gyrus (BA6, ventrolateral)-Rt. precentral gyrus (BA4, upper limb) |
| #27 | Rt. superior parietal lobule (BA5, lateral)-Rt. inferior parietal lobule (BA40, |
| | rostroventral) |
| #28 | Lt. superior parietal lobule (BA5, intraparietal)-Rt. parietooccipital sulcus |
| | (ventromedial) |
| #29 | Rt. superior temporal gyrus (BA41/42)-Lt. thalamus (rostral temporal) |

TABLE 2

| Rank (#i) | Brain functional connectivity region |
|---|---|
| #30 | Rt. superior temporal gyrus (BA38, medial)-Rt. cerebellum (lobule VI) |
| #31 | Rt. superior temporal gyrus (BA38, medial)-Rt. lingual gyrus (rostral) |
| #32 | Rt. superior temporal gyrus (BA38, medial)-Rt. parahippocampal gyrus (area TL) |
| #33 | Lt. superior temporal gyrus (BA22, caudal)-Rt. superior temporal gyrus (BA22, |
| | rostral) |
| #34 | Lt. cerebellum (lobule IX)-Brainstem |
| #35 | Lt. parahippocampal gyrus (BA35/36, rostral)-Vermis. cerebellum (lobule IX) |
| #36 | Rt. superior temporal gyrus (BA38, medial)-Lt. inferior temporal gyrus (BA20, |
| | rostral) |
| #37 | Rt. superior temporal gyrus (BA38, medial)-Rt. parietooccipital sulcus |
| | (ventromedial) |
| #38 | Lt. precentral gyrus (BA4, head and face)-Lt. inferior temporal gyrus (BA20, rostral) |
| #39 | Lt. inferior temporal gyrus (BA20, rostral)-Lt. cingulate (BA23, caudal) |

2. A system for diagnosing sustained pain, comprising:
a receiver for receiving brain image data of a subject;
an analyzer for analyzing one or more selected from 1 to 39 brain functional connectivity regions listed in Tables 1 and 2 below from the received data; and
a calculator for calculating a signature response based on the brain functional connectivity.
TABLE 1

| Rank (#i) | Brain functional connectivity region |
|---|---|
| #1 | Lt. Inferior temporal gyrus (BA37, ventrolateral)-Lt. middle occipital gyrus |
| #2 | Lt. middle temporal gyrus (BA37, dorsolateral)-Lt. middle occipital gyrus |
| #3 | Lt. precentral gyrus (BA4, trunk)-Rt. precuneus (BA7, medial) |
| #4 | Lt. precuneus (BA7, medial)-Lt. postcentral gyrus (BA1/2/3, trunk) |
| #5 | Rt. inferior parietal lobule (BA40, rostrodorsal)-Lt. parietooccipital sulcus |
| | (dorsomedial) |
| #6 | Rt. precentral gyrus (BA4, upper limb)-Lt. inferior parietal lobule (BA39, |
| | rostrodorsal) |
| #7 | Lt. precentral gyrus (BA4, trunk)-Rt. medial superior occipital gyrus |
| #8 | Lt. paracentral lobule (BA4, lower limb)-Rt. precuneus (BA8, medial) |
| #9 | Lt. precentral gyrus (BA4, trunk)-Lt. precuneus (BA8, medial) |
| #10 | Rt. precuneus (BA7, medial)-Lt. postcentral gyrus (BA1/2/3, trunk) |
| #11 | Lt. superior temporal gyrus (BA22, rostral)-Lt. middle temporal gyrus (BA37, |
| | dorsolateral) |
| #12 | Lt. precentral gyrus (BA4, trunk)-Lt. superior parietal lobule (BA7, rostral) |
| #13 | Lt. paracentral lobule (BA4, lower limb)-Lt. inferior temporal gyrus (BA37, |
| | ventrolateral) |
| #14 | Rt. posterior superior temporal sulcus (caudal)-Rt. inferior parietal lobule (BA39, |
| | rostrodorsal) |
| #15 | Rt. paracentral lobule (BA4, lower limb)-Rt. lingual gyrus (caudal) |
| #16 | Rt. inferior parietal lobule (BA40, rostroventral)-Lt. precuneus (BA7, medial) |
| #17 | Rt. superior temporal gyrus (BA41/42)-Lt. caudate (dorsal) |
| #18 | Rt. posterior superior temporal sulcus (caudal)-Hypothalamus |
| #19 | Lt. precentral gyrus (BA4, trunk)-Rt. superior parietal lobule (BA7, caudal) |
| #20 | Lt. inferior parietal lobule (BA39, rostrodorsal)-Lt. medial superior occipital gyrus |
| #21 | Lt. superior parietal lobule (BA7, postcentral)-Lt. inferior parietal lobule (BA39, |
| | caudal) |
| #22 | Rt. paracentral lobule (BA4, lower limb)-Rt. inferior occipital gyrus |
| #23 | Rt. precuneus (BA5, medial)-Lt. postcentral gyrus (BA1/2/3, trunk) |
| #24 | Rt. precuneus (BA5, medial)-Lt. VS/MT+ |
| #25 | Rt. superior temporal gyrus (BA41/42)-Lt. thalamus (caudal temporal) |
| #26 | Lt. middle frontal gyrus (BA6, ventrolateral)-Rt. precentral gyrus (BA4, upper limb) |
| #27 | Rt. superior parietal lobule (BA5, lateral)-Rt. inferior parietal lobule (BA40, |
| | rostroventral) |
| #28 | Lt. superior parietal lobule (BA5, intraparietal)-Rt. parietooccipital sulcus |
| | (ventromedial) |
| #29 | Rt. superior temporal gyrus (BA41/42)-Lt. thalamus (rostral temporal) |

TABLE 2

| Rank (#i) | Brain functional connectivity region |
|---|---|
| #30 | Rt. superior temporal gyrus (BA38, medial)-Rt. cerebellum (lobule VI) |
| #31 | Rt. superior temporal gyrus (BA38, medial)-Rt. lingual gyrus (rostral) |
| #32 | Rt. superior temporal gyrus (BA38, medial)-Rt. parahippocampal gyrus (area TL) |
| #33 | Lt. superior temporal gyrus (BA22, caudal)-Rt. superior temporal gyrus (BA22, |
| | rostral) |
| #34 | Lt. cerebellum (lobule IX)-Brainstem |
| #35 | Lt. parahippocampal gyrus (BA35/36, rostral)-Vermis. cerebellum (lobule IX) |
| #36 | Rt. superior temporal gyrus (BA38, medial)-Lt. inferior temporal gyrus (BA20, |
| | rostral) |
| #37 | Rt. superior temporal gyrus (BA38, medial)-Rt. parietooccipital sulcus |
| | (ventromedial) |
| #38 | Lt. precentral gyrus (BA4, head and face)-Lt. inferior temporal gyrus (BA20, rostral) |
| #39 | Lt. inferior temporal gyrus (BA20, rostral)-Lt. cingulate (BA23, caudal) |

3. The system of claim 2, wherein the brain image data is MRI data.

4. The system of claim 2, wherein the signature response is calculated by Equation 1 below:
Signature response= [symbol] · [symbol] =Σᵢ₌₁ⁿwᵢxᵢ. [Equation 1]
(Here, n is an integer of 1 to 39,
i is an integer of n or less,
wᵢ is a weight corresponding to the brain functional connectivity of #i, and xᵢ is test data corresponding to the brain functional connectivity of #i).

5. The system of claim 4, wherein the wᵢ is a weight corresponding to the brain functional connectivity of #i, listed in Tables 3 and 4 below:
TABLE 3

| Rank | |
|---|---|
| (#i) | Weights |
| #1 | 0.0003308 |
| #2 | 0.0003259 |
| #3 | 0.0002891 |
| #4 | 0.0002799 |
| #5 | 0.0002773 |
| #6 | 0.0002771 |
| #7 | 0.0002635 |
| #8 | 0.0002634 |
| #9 | 0.0002541 |
| #10 | 0.0002474 |
| #11 | 0.0002400 |
| #12 | 0.0002322 |
| #13 | 0.0002285 |
| #14 | 0.0002265 |
| #15 | 0.0002182 |
| #16 | 0.0002085 |
| #17 | 0.0001976 |
| #18 | 0.0001834 |
| #19 | 0.0001829 |
| #20 | 0.0001754 |
| #21 | 0.0001744 |
| #22 | 0.0001726 |
| #23 | 0.0001654 |
| #24 | 0.0001625 |
| #25 | 0.0001584 |
| #26 | 0.0001518 |
| #27 | 0.0001333 |
| #28 | 0.0001313 |
| #29 | 0.0001304 |

TABLE 4

| Rank | |
|---|---|
| (#i) | Weights |
| #30 | −0.0004139 |
| #31 | −0.0003892 |
| #32 | −0.0003209 |
| #33 | −0.0003077 |
| #34 | −0.0003033 |
| #35 | −0.0003008 |
| #36 | −0.0002895 |
| #37 | −0.0002862 |
| #38 | −0.0002532 |
| #39 | −0.0001850 |

6. The system of claim 2, wherein the sustained pain lasts for 10 seconds or more.

7. A method for diagnosing sustained pain, comprising:
applying a stimulus to an individual and receiving brain image data according to the stimulus;
analyzing one or more selected from 1 to 39 brain functional connectivity listed in Tables 1 and 2 below; and
calculating a signature response based on the brain functional connectivity.
TABLE 1

| Rank (#i) | Brain functional connectivity region |
|---|---|
| #1 | Lt. Inferior temporal gyrus (BA37, ventrolateral)-Lt. middle occipital gyrus |
| #2 | Lt. middle temporal gyrus (BA37, dorsolateral)-Lt. middle occipital gyrus |
| #3 | Lt. precentral gyrus (BA4, trunk)-Rt. precuneus (BA7, medial) |
| #4 | Lt. precuneus (BA7, medial)-Lt. postcentral gyrus (BA1/2/3, trunk) |
| #5 | Rt. inferior parietal lobule (BA40, rostrodorsal)-Lt. parietooccipital sulcus |
| | (dorsomedial) |
| #6 | Rt. precentral gyrus (BA4, upper limb)-Lt. inferior parietal lobule (BA39, |
| | rostrodorsal) |
| #7 | Lt. precentral gyrus (BA4, trunk)-Rt. medial superior occipital gyrus |
| #8 | Lt. paracentral lobule (BA4, lower limb)-Rt. precuneus (BA8, medial) |
| #9 | Lt. precentral gyrus (BA4, trunk)-Lt. precuneus (BA8, medial) |
| #10 | Rt. precuneus (BA7, medial)-Lt. postcentral gyrus (BA1/2/3, trunk) |
| #11 | Lt. superior temporal gyrus (BA22, rostral)-Lt. middle temporal gyrus (BA37, |
| | dorsolateral) |
| #12 | Lt. precentral gyrus (BA4, trunk)-Lt. superior parietal lobule (BA7, rostral) |
| #13 | Lt. paracentral lobule (BA4, lower limb)-Lt. inferior temporal gyrus (BA37, |
| | ventrolateral) |
| #14 | Rt. posterior superior temporal sulcus (caudal)-Rt. inferior parietal lobule (BA39, |
| | rostrodorsal) |
| #15 | Rt. paracentral lobule (BA4, lower limb)-Rt. lingual gyrus (caudal) |
| #16 | Rt. inferior parietal lobule (BA40, rostroventral)-Lt. precuneus (BA7, medial) |
| #17 | Rt. superior temporal gyrus (BA41/42)-Lt. caudate (dorsal) |
| #18 | Rt. posterior superior temporal sulcus (caudal)-Hypothalamus |
| #19 | Lt. precentral gyrus (BA4, trunk)-Rt. superior parietal lobule (BA7, caudal) |
| #20 | Lt. inferior parietal lobule (BA39, rostrodorsal)-Lt. medial superior occipital gyrus |
| #21 | Lt. superior parietal lobule (BA7, postcentral)-Lt. inferior parietal lobule (BA39, |
| | caudal) |
| #22 | Rt. paracentral lobule (BA4, lower limb)-Rt. inferior occipital gyrus |
| #23 | Rt. precuneus (BA5, medial)-Lt. postcentral gyrus (BA1/2/3, trunk) |
| #24 | Rt. precuneus (BA5, medial)-Lt. VS/MT+ |
| #25 | Rt. superior temporal gyrus (BA41/42)-Lt. thalamus (caudal temporal) |
| #26 | Lt. middle frontal gyrus (BA6, ventrolateral)-Rt. precentral gyrus (BA4, upper limb) |
| #27 | Rt. superior parietal lobule (BA5, lateral)-Rt. inferior parietal lobule (BA40, |
| | rostroventral) |
| #28 | Lt. superior parietal lobule (BA5, intraparietal)-Rt. parietooccipital sulcus |
| | (ventromedial) |
| #29 | Rt. superior temporal gyrus (BA41/42)-Lt. thalamus (rostral temporal) |

TABLE 2

| Rank (#i) | Brain functional connectivity region |
|---|---|
| #30 | Rt. superior temporal gyrus (BA38, medial)-Rt. cerebellum (lobule VI) |
| #31 | Rt. superior temporal gyrus (BA38, medial)-Rt. lingual gyrus (rostral) |
| #32 | Rt. superior temporal gyrus (BA38, medial)-Rt. parahippocampal gyrus (area TL) |
| #33 | Lt. superior temporal gyrus (BA22, caudal)-Rt. superior temporal gyrus (BA22, |
| | rostral) |
| #34 | Lt. cerebellum (lobule IX)-Brainstem |
| #35 | Lt. parahippocampal gyrus (BA35/36, rostral)-Vermis. cerebellum (lobule IX) |
| #36 | Rt. superior temporal gyrus (BA38, medial)-Lt. inferior temporal gyrus (BA20, |
| | rostral) |
| #37 | Rt. superior temporal gyrus (BA38, medial)-Rt. parietooccipital sulcus |
| | (ventromedial) |
| #38 | Lt. precentral gyrus (BA4, head and face)-Lt. inferior temporal gyrus (BA20, rostral) |
| #39 | Lt. inferior temporal gyrus (BA20, rostral)-Lt. cingulate (BA23, caudal) |

8. The method of claim 7, comprising:
determining that a subject feels pain the more the connectivity in Table 1 is present.

9. The method of claim 7, comprising:
determining that a subject feels pain the less the connectivity in Table 2 is present.

10. The method of claim 7, wherein the signature response is calculated by Equation 1 below:
Signature response= [symbol] · [symbol] =Σᵢ₌₁ⁿwᵢxᵢ. [Equation 1]
(Here, n is an integer of 1 to 39,
i is an integer of n or less,
wᵢ is a weight corresponding to the brain functional connectivity of #i, and xᵢ is test data corresponding to the brain functional connectivity of #i).

11. A sustained pain diagnosis model in which it is determined that the higher signature response calculated by Equation 1 below the more sustained pain a subject feels:
Signature response= [symbol] · [symbol] =Σᵢ₌₁ⁿwᵢxᵢ. [Equation 1]
(Here, n is an integer of 1 to 39,
i is an integer of n or less,
wᵢ is a weight corresponding to the brain functional connectivity of #i, and xᵢ is test data corresponding to the brain functional connectivity of #i).

12. A system for diagnosing sustained pain and determining the effect of relieving the pain, comprising:
a receiver for receiving brain image data of a subject;
an analyzer for analyzing one or more selected from 1 to 39 brain functional connectivity regions listed in Tables 1 and 2 below from the received data; and
a calculator for calculating a signature response based on the brain functional connectivity.
TABLE 1

| Rank (#i) | Brain functional connectivity region |
|---|---|
| #1 | Lt. Inferior temporal gyrus (BA37, ventrolateral)-Lt. middle occipital gyrus |
| #2 | Lt. middle temporal gyrus (BA37, dorsolateral)-Lt. middle occipital gyrus |
| #3 | Lt. precentral gyrus (BA4, trunk)-Rt. precuneus (BA7, medial) |
| #4 | Lt. precuneus (BA7, medial)-Lt. postcentral gyrus (BA1/2/3, trunk) |
| #5 | Rt. inferior parietal lobule (BA40, rostrodorsal)-Lt. parietooccipital sulcus |
| | (dorsomedial) |
| #6 | Rt. precentral gyrus (BA4, upper limb)-Lt. inferior parietal lobule (BA39, |
| | rostrodorsal) |
| #7 | Lt. precentral gyrus (BA4, trunk)-Rt. medial superior occipital gyrus |
| #8 | Lt. paracentral lobule (BA4, lower limb)-Rt. precuneus (BA8, medial) |
| #9 | Lt. precentral gyrus (BA4, trunk)-Lt. precuneus (BA8, medial) |
| #10 | Rt. precuneus (BA7, medial)-Lt. postcentral gyrus (BA1/2/3, trunk) |
| #11 | Lt. superior temporal gyrus (BA22, rostral)-Lt. middle temporal gyrus (BA37, |
| | dorsolateral) |
| #12 | Lt. precentral gyrus (BA4, trunk)-Lt. superior parietal lobule (BA7, rostral) |
| #13 | Lt. paracentral lobule (BA4, lower limb)-Lt. inferior temporal gyrus (BA37, |
| | ventrolateral) |
| #14 | Rt. posterior superior temporal sulcus (caudal)-Rt. inferior parietal lobule (BA39, |
| | rostrodorsal) |
| #15 | Rt. paracentral lobule (BA4, lower limb)-Rt. lingual gyrus (caudal) |
| #16 | Rt. inferior parietal lobule (BA40, rostroventral)-Lt. precuneus (BA7, medial) |
| #17 | Rt. superior temporal gyrus (BA41/42)-Lt. caudate (dorsal) |
| #18 | Rt. posterior superior temporal sulcus (caudal)-Hypothalamus |
| #19 | Lt. precentral gyrus (BA4, trunk)-Rt. superior parietal lobule (BA7, caudal) |
| #20 | Lt. inferior parietal lobule (BA39, rostrodorsal)-Lt. medial superior occipital gyrus |
| #21 | Lt. superior parietal lobule (BA7, postcentral)-Lt. inferior parietal lobule (BA39, |
| | caudal) |
| #22 | Rt. paracentral lobule (BA4, lower limb)-Rt. inferior occipital gyrus |
| #23 | Rt. precuneus (BA5, medial)-Lt. postcentral gyrus (BA1/2/3, trunk) |
| #24 | Rt. precuneus (BA5, medial)-Lt. VS/MT+ |
| #25 | Rt. superior temporal gyrus (BA41/42)-Lt. thalamus (caudal temporal) |
| #26 | Lt. middle frontal gyrus (BA6, ventrolateral)-Rt. precentral gyrus (BA4, upper |
| | limb) |
| #27 | Rt. superior parietal lobule (BA5, lateral)-Rt. inferior parietal lobule (BA40, |
| | rostroventral) |
| #28 | Lt. superior parietal lobule (BA5, intraparietal)-Rt. parietooccipital sulcus |
| | (ventromedial) |
| #29 | Rt. superior temporal gyrus (BA41/42)-Lt. thalamus (rostral temporal) |

TABLE 2

| Rank (#i) | Brain functional connectivity region |
|---|---|
| #30 | Rt. superior temporal gyrus (BA38, medial)-Rt. cerebellum (lobule VI) |
| #31 | Rt. superior temporal gyrus (BA38, medial)-Rt. lingual gyrus (rostral) |
| #32 | Rt. superior temporal gyrus (BA38, medial)-Rt. parahippocampal gyrus (area TL) |
| #33 | Lt. superior temporal gyrus (BA22, caudal)-Rt. superior temporal gyrus (BA22, |
| | rostral) |
| #34 | Lt. cerebellum (lobule IX)-Brainstem |
| #35 | Lt. parahippocampal gyrus (BA35/36, rostral)-Vermis. cerebellum (lobule IX) |
| #36 | Rt. superior temporal gyrus (BA38, medial)-Lt. inferior temporal gyrus (BA20, |
| | rostral) |
| #37 | Rt. superior temporal gyrus (BA38, medial)-Rt. parietooccipital sulcus |
| | (ventromedial) |
| #38 | Lt. precentral gyrus (BA4, head and face)-Lt. inferior temporal gyrus (BA20, rostral) |
| #39 | Lt. inferior temporal gyrus (BA20, rostral)-Lt. cingulate (BA23, caudal) |
